(19) United States
(12) Patent Application Publication
(10) Pub. No.: US 2026/0221273 A1
(43) Pub. Date: Jul. 30, 2026

(54) COHESIVE MOBILE APPLICATION FOR RESIDENTIAL CARE

(71) Applicant: MatrixCare, Inc., Bloomington, MN (US)

(72) Inventors: Lee Eric KILMER, Stillwater, MN (US); Heitor Amaro BARCELLOS NETO, Sao Paulo (BR); Aneesur RAHMAN, Maple Grove, MN (US); Niharika MEHTA, Bangalore (IN)

(21) Appl. No.: 19/447,274

(22) Filed: Jan. 13, 2026

Related U.S. Application Data
(60) Provisional application No. 63/751,415, filed on Jan. 30, 2025.

(51) Int. Cl. G16H 40/20; G16H 10/60

(52) U.S. Cl. CPC G16H 40/20; G16H 10/60

(57) ABSTRACT

Techniques for improved data access and management are provided. An indication of a resident of a residential care facility is received via an application executing on a client device. An activity timeline corresponding to the resident is accessed, where the activity timeline comprises a sequence of healthcare events for the resident. The activity timeline is provided for output via the application, and an inquiry about a healthcare event on the activity timeline is received via the application. In response to receiving the inquiry, a caregiver assigned to the resident at a current time is identified. The inquiry is forwarded to the caregiver, and a response from the caregiver is provided for output via the application.

### CROSS-REFERENCE TO RELATED APPLICATIONS

This application claims priority to U.S. Provisional Patent Application No. 63/751,415, filed January 30, 2025, the entire content of which is incorporated herein by reference in its entirety.

### TECHNICAL FIELD

The present disclosure relates generally to residential care, and more particularly, to mobile applications to improve residential care.

A wide variety of healthcare services are provided via residential or in-home care, such as when a patient resides in a long-term care facility. These residential facilities have become increasingly popular, particularly with rising senior populations and demographic shifts. However, with this increasing number of residents, the burden on caregivers has similarly increased. A large amount of resources are expended providing non-substantive care. For example, it has been estimated that nursing staff in a residential care facility currently spend over three hours per day communicating with family members of residents, (e.g., through phone calls, personal visits, and the like). This is a significant contributor to clinician burnout. Similarly, caregivers often spend significant time (e.g., from a third to a half of each shift) on documentation tasks, including recording updates, retrieving information about updates that occurred prior to the caregiver beginning their shift (e.g., overnight or from the previous day), and the like.

For similar reasons, friends and families of residents often find themselves overwhelmed and disconnected from their loved ones in such residential communities, and it has become increasingly difficult to stay abreast of a loved one’s condition without investing significant time and effort to personally track down the desired information. Currently, the relevant information is often distributed across a wide variety of repositories, each often having different security and access policies, rendering data access and updates opaque and difficult.

### SUMMARY

According to some implementations of the present disclosure, a method includes: receiving, via an application executing on a client device, an indication of a first resident of a residential care facility; accessing a first activity timeline corresponding to the first resident, wherein the first activity timeline comprises a sequence of healthcare events for the first resident; providing the first activity timeline for output via the application; receiving, via the application, a first inquiry about a first healthcare event on the first activity timeline; in response to receiving the first inquiry, identifying a first caregiver assigned to the first resident at a current time; forwarding the first inquiry to the first caregiver; and providing a first response from the first caregiver for output via the application.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 depicts an example environment for improved care and data synchronization, according to some embodiments of the present disclosure.

FIG. 2 depicts an example interface summarizing patient information by synchronizing across sources, according to some embodiments of the present disclosure.

FIG. 3 depicts an example interface depicting a unified activity timeline from disparate record sources, according to some embodiments of the present disclosure.

FIG. 4 depicts an example interface to provide secure chat functionality, according to some embodiments of the present disclosure.

FIG. 5 depicts an example interface to provide secure chat functionality with caregivers, according to some embodiments of the present disclosure.

FIG. 6 depicts an example interface to provide secure chat functionality with machine learning-based systems, according to some embodiments of the present disclosure.

FIG. 7 depicts an example interface for assignment selection, according to some embodiments of the present disclosure.

FIG. 8 depicts an example interface for resident selection, according to some embodiments of the present disclosure.

FIG. 9 depicts an example interface for evaluating synchronized data across residents, according to some embodiments of the present disclosure.

FIG. 10 is a flow diagram depicting an example method for synchronizing data and providing cohesive updates, according to some embodiments of the present disclosure.

FIG. 11 is a flow diagram depicting an example method for enabling user interaction with data systems, according to some embodiments of the present disclosure.

FIG. 12 is a flow diagram depicting an example method for secure document management, according to some embodiments of the present disclosure.

FIG. 13 is a flow diagram depicting an example method for secure chat interactions, according to some embodiments of the present disclosure.

FIG. 14 is a flow diagram depicting an example method for improved healthcare, according to some embodiments of the present disclosure.

FIG. 15 depicts an example computing device configured to perform various aspects of the present disclosure, according to some embodiments disclosed herein.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure generally provide techniques for data synchronization using a cohesive healthcare application to unify disparate sources and systems to improve residential care and reduce data access and storage costs.

In some embodiments, the healthcare application provides a mobile-first improvement to healthcare management for residents and families, while providing significant efficiencies that reduces the burden on caregivers and family members while improving data access and understanding. In some aspects, the described applications can unify multiple disparate aspects of residential care that currently rely on multiple systems and workflows, introducing inherent inefficiency. For example, in some embodiments, the centralized application can provide seamless access to (and organization of) medical records of the resident via a comprehensible in-application interface, allowing users (e.g., family members, loved ones, clinicians, caregivers, and the residents themselves) to easily access and review such records at any time. In some embodiments, the application can further manage secure documentation, such as requests for signature (e.g., of the resident or of a designated family member or friend), seamlessly within the application itself (rather than relying on extrinsic systems). Further, in some embodiments, the application can enable effortless communication between users (e.g., family members) and caregivers in a way that reduces error and miscommunication while meeting security standards (e.g., using communication channels that are Health Insurance Portability and Accountability Act (HIPAA)) compliant).

In these ways, the described techniques can significantly improve residential care while reducing the effort, potential for error introduction, and computational expense of accessing discrete systems for each desired task. For example, easily accessible health information for caregivers and family members via activity timelines can significantly reduce the time and expense of manually seeking out these updates. Consolidated communication and notification channels via automated chat routing can ensure HIPAA compliance with reduced computational overhead.

Generally, aspects of the present disclosure simplify data workflows by consolidating and synchronizing discrete systems via a global application that can manage and improve all aspects of residential care. Advantageously, by using embodiments of the present disclosure, caregivers are able to better organize and access disparate records and systems (which may otherwise be incompatible) using a unified interface, allowing the application systems to handle any relevant operations needed to join the data systems, which leaves caregivers free to focus on residential care itself. Similarly, by using embodiments of the present disclosure, users (e.g., family members and friends of residents or patients) are able to readily access health records that are otherwise inaccessible (or difficult to access), while also providing streamline secure document review and easy interactivity with caregivers, all within a unified application.

### Example Environment for Improved Care and Data Synchronization

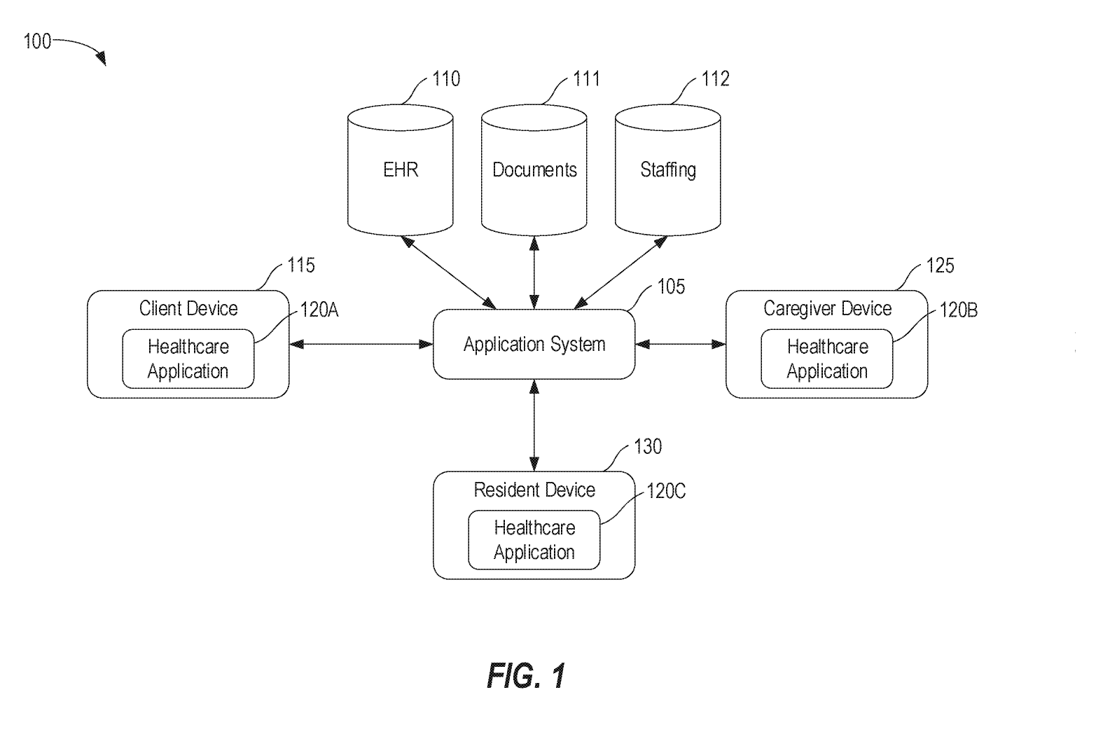

FIG. 1 depicts an example environment 100 for improved care and data synchronization, according to some embodiments of the present disclosure.

In the illustrated environment 100, an application system 105 is communicatively coupled with a variety of other devices, including a client device 115, a caregiver device 125, and a resident device 130. In some embodiments, the client device 115, caregiver device 125, and resident device 130 may collectively be referred to as “user devices.” In the illustrated example, the client device 115, caregiver device 125, and resident device 130 are each generally representative of a personal computing system used to interact with the application system 105. For example, each of the client device 115, the caregiver device 125, and the resident device 130 may correspond to smartphones, laptop computers, tablets, wearables, and the like. Generally, the client device 115 may be associated with (e.g., used by) a user such as a family member or friend of a resident or patient in a residential care facility. The caregiver device 125 may be associated with (e.g., used by) a caregiver in a one or more residential facilities, such as a nurse, clinician, doctor, and the like. The resident device 130 may be associated with (e.g., used by) a resident or patient in a residential care facility. Although a single client device 115, a single caregiver device 125, and a single resident device 130 are depicted for conceptual clarity, in some aspects, there may be any number and variety of the client devices 115 (e.g., for multiple family members of any number of patients), caregiver devices 125 (e.g., for multiple caregivers in one or more facilities), and/or resident devices 130 (e.g., for multiple residents in one or more facilities).

In the illustrated example, each of the client device 115, the caregiver device 125, and the resident device 130 are hosting or executing a corresponding instance of a healthcare application 120. Specifically, the client device 115 is executing the healthcare application 120A, the caregiver device 125 is executing the healthcare application 120B, and the resident device 130 is executing the healthcare application 120C. The healthcare application 120 executing on each device is generally representative of a software application that enables a wide variety of functionality discussed above and below, facilitated by the application system 105. In some embodiments, the user of a given device can log in or otherwise authenticate themselves with the healthcare application 120, allowing the healthcare application 120 (or the application system 105) to determine the role of the user (e.g., whether they are a resident, a caregiver, or a support person of a resident), the resident(s) associated with the user, and the like.

In the illustrated example, the application system 105 is generally representative of any computing system capable of performing the various embodiments described. Although depicted as a discrete computing system for conceptual clarity, the operations of the application system 105 may be combined or distributed across any number and variety of systems, and may generally be implemented using hardware, software, or a combination of hardware and software. Generally, the application system 105 may be communicatively coupled with the healthcare application 120 executing on each device using any combination of wired and wireless links. In some embodiments, the application system 105 is implemented as a server system or cloud-based service, and the application system 105 interacts with the healthcare applications 120 via the Internet. In some embodiments, some or all of the operations of the application system 105 may be implemented by the healthcare applications 120 themselves. Similarly, in some embodiments, some or all of the operations of the healthcare applications 120 may be performed by the application system 105.

In the illustrated example, the application system 105 can access a variety of repositories, including a repository of electronic health records (EHR) 110, a repository of secure documents 111, and a repository with information relating to staffing 112. Although depicted as three discrete repositories, in some embodiments, the data stored in the EHR 110, documents 111, and staffing 112 may be stored across any number and variety of repositories.

In some embodiments, the EHR 110 generally comprises electronic health records (e.g., medical records) for one or more patients or residents in one or more residential care facilities. For example, the EHR 110 may include, for any number of residents, records indicating data such as daily check-in information (e.g., notes written by a caregiver), vitals recorded throughout the day (e.g., blood pressure, heart rate, temperature, and the like), records relating to interactions between the resident and clinicians, records of medication(s) the resident uses, records of diagnoses the resident has received, and the like.

In some embodiments, the EHR 110 may generally be maintained in a secure or protected environment, such that users without authorization cannot readily access the data. In some embodiments, the application system 105 can seamlessly and automatically manage user access to the EHR 110, which may include allowing authorized caregivers (e.g., using the healthcare application 120B executing on their caregiver device 125) to update or add new records to the EHR 110 as relevant (e.g., updating the medications, clinician notes, diagnoses, and the like). Similarly, the application system 105 may allow users (via the healthcare application 120A on the client device 115) to view the relevant records for which the user has authorized access (e.g., for the resident(s) that the user has authorization). For example, as discussed above, the user may authenticate themselves with the healthcare application 120A, and the healthcare application 120A may thereafter selectively retrieve the secure EHR 110 to which the user is entitled access, without exposing other records and/or without letting the user themselves specify the record(s) they desire.

In some embodiments, as discussed in more detail below, the application system 105 may access the EHR 110 of a given resident and may dynamically generate an activity timeline depicting the various updates or changes over time. By outputting this activity timeline for display via the healthcare application 120A (e.g., via a graphical user interface (GUI)), the application system 105 can allow the user to readily review the resident’s health over time, as well as quickly identifying any updates that may have occurred. In some embodiments, when updated records are available (e.g., a new set of medications added a new diagnosis, a new clinician note, and the like), the application system 105 may automatically retrieve these updates and update the activity timeline. In some embodiments, the application system 105 may update the activity timeline in response to user request (e.g., when the user logs into the healthcare application 120A or requests an updated timeline).

In some embodiments, the documents 111 generally comprises secure medical documents for one or more patients or residents in one or more residential care facilities. For example, the documents 111 may include, for any number of residents, documents such as intake forms, consent forms, disclosure forms, and the like. In some embodiments, the documents 111 generally include documents that require a user signature (e.g., from the resident or from an associate of the resident, such as a family member having healthcare power of attorney). For example, in some embodiments, the documents 111 may include informed consent documents where, by signing, the executor attests that they understand the contents of the document, such as the potential risks of a surgery or other procedure. In some embodiments, the documents 111 similarly store signed documents in accordance with various regulations or policies.

In some embodiments, the documents 111 may generally be maintained in a secure or protected environment, such that users without authorization cannot access the data. In some embodiments, the application system 105 can seamlessly and automatically manage user access to the documents 111, which may include allowing authorized caregivers (e.g., using the healthcare application 120B executing on their caregiver device 125) to update or add new documents (or to flag needed signatures) to the documents 111. Similarly, the application system 105 may allow users (via the healthcare application 120A on the client device 115) to view the relevant documents for which the user has authorized access (e.g., for the resident(s) that the user has authorization). For example, as discussed above, the user may authenticate themselves with the healthcare application 120A, and the healthcare application 120A may thereafter selectively retrieve the secure documents 111 to which the user is entitled access, without exposing other records and/or without letting the user themselves specify the record(s) they desire.

In some embodiments, as discussed in more detail below, the application system 105 may access the documents 111 and dynamically generate alerts or notifications (which, in some embodiments, may be included on the activity timeline of the resident) indicating the documents 111 that require attention (e.g., that are awaiting the user’s review and signature). In some embodiments, the application system 105 may use an integrated electronic signature functionality to allow users to sign the document(s) directly within the healthcare application 120A. That is, the user may retrieve, review, and sign the document within the healthcare application 120A itself, rather than relying on external or independent document management applications or systems. This unification of the documents 111 within the application system 105 can significantly reduce the burden and computational expense of the document signing process. For example, the user need not exit the healthcare application 120A to open a document signing application (which would incur additional memory overhead and latency to switch compute contexts).

In some embodiments, the staffing 112 generally comprises staffing-related information for one or more residents and/or caregivers in one or more residential care facilities. For example, the staffing 112 may indicate, for any number of care facilities, which caregiver(s) are assigned to each facility, to each unit, and/or to each resident or other task or job at any given time. For example, in some embodiments, the staffing 112 may indicate which nurse(s) are tasked with caring for a given set of residents in a given facility on a given day.

In some embodiments, the staffing 112 may generally be maintained in a secure or protected environment, such that users do not have access to the data. For example, a user of the healthcare application 120A may not be able to view which nurse(s) are on staff at any given time. In some embodiments, as discussed in more detail below, the application system 105 may utilize the staffing 112 to dynamically route interactions or communications (e.g., chat requests). For example, the user may (via the healthcare application 120A on the client device 115) provide a request or inquiry relating to the care of a resident associated with the user. In some embodiments, the application system 105 may identify or determine the context of the request (e.g., determining whether the user is asking about ongoing medical care, financial needs, therapy, social services, records or documents, activities, dietary concerns, and the like). Based on this context, the application system 105 may evaluate the staffing 112 to determine which user(s) should be tapped to respond to the inquiry (e.g., ensuring that a question about a new diagnosis are routed to the clinician that provided the diagnosis, or ensuring that a question about the current state of the resident is routed to the current nurse or other caregiver assigned to the resident).

Advantageously, by combining and managing access to these disparate systems and repositories, the application system 105 can streamline the data collection and analysis processes and enable far improved data access. For example, the relevant information distributed across repositories (e.g., the EHR 110 and documents 111) may otherwise be difficult or impossible for an average user to access (without relying on requesting that a more experienced or authorized individual, such as a nurse, manually retrieve the information). Further, using dynamic authorization or authentication, the application system 105 can ensure (via the healthcare applications 120) that the users are able to access only authorized information, improving data security. Further, by utilizing the staffing 112, the application system 105 can ensure that HIPAA requirements are met without introducing burdensome or frustrating communication blockages.

Further, as discussed in more detail below, the unified interfaces provided using the application system 105 can substantially improve other aspects, including the health and safety of residents. For example, by providing seamless updates to users and caregivers regarding resident statuses and healthcare events, the application system can ensure that residents receive particularized care precisely the care is needed. Similarly, by aggregating updates from disparate repositories and providing unified summaries to users, care is improved. Moreover, automatic chat evaluations to determine whether to use generative machine learning to respond to inquiries can ensure highly accurate and reliable information is provided to users as quickly as possible, reducing potential delays in care provisioning.

### Example Interface Summarizing Patient Information by Synchronizing Across Sources

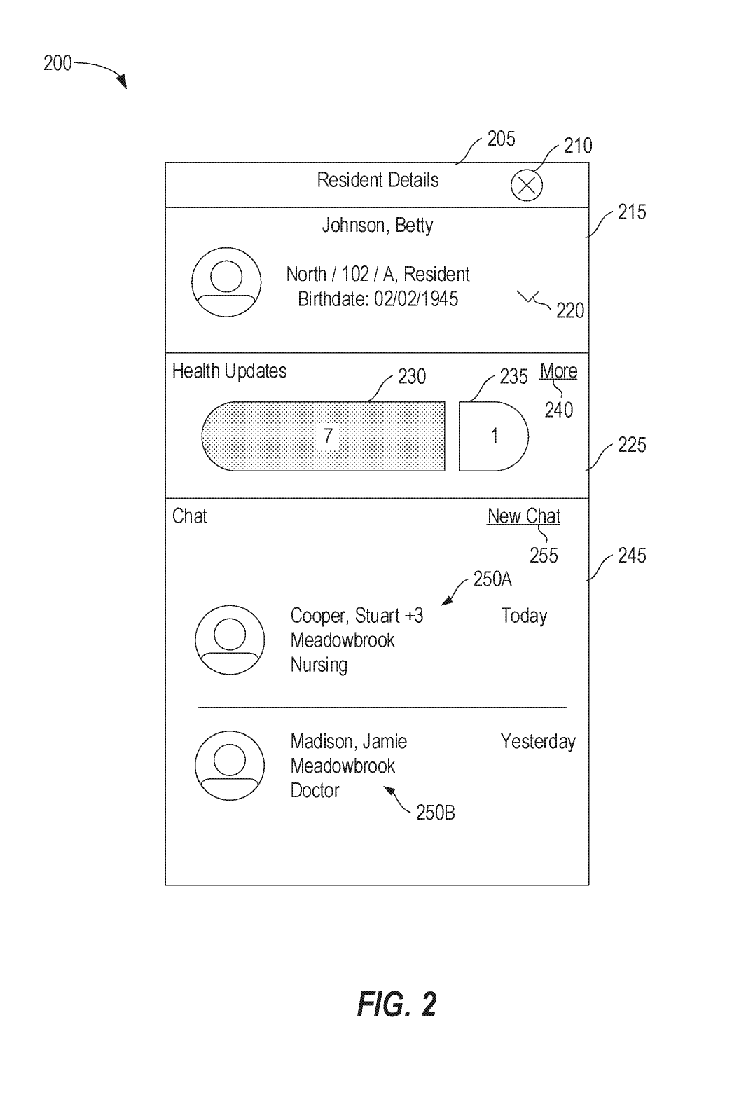

FIG. 2 depicts an example interface 200 summarizing patient information by synchronizing across sources, according to some embodiments of the present disclosure. In some embodiments, the interface 200 is a GUI of a healthcare application, such as the healthcare application 120 of FIG. 1. For example, the illustrated interface 200 may correspond to the healthcare application 120A executing on a client device 115, each of FIG. 1, where the contents of the interface 200 are generated or facilitated by an application system, such as the application system 105 of FIG. 1.

In the illustrated example, the interface is displaying resident details 205 of a resident (e.g., a patient) in a residential care facility. For example, in some embodiments, upon the user opening the healthcare application and/or authenticating (e.g., logging in using a username and password, using a biometric passcode such as a fingerprint or face scan, and the like), the healthcare application may identify any resident(s) associated with the user. For example, the healthcare application may determine whether the user is enrolled as a trusted friend or family member of one or more residents registered with the healthcare application. In some embodiments, when a resident is registered (e.g., by themselves or with assistance from a caregiver), the “trusted” individuals who should have access to the resident’s information via the healthcare application may specified, allowing such individuals to later enroll with the healthcare application and access such information.

In some embodiments, if a single user is associated with multiple residents, the healthcare application may present a list of the available residents, and the user may select which resident they wish to see in more detail. In the illustrated example, the icon 210 may be used to close or exit the current resident’s profile (e.g., to see other profiles, to see a home page or welcome screen for the healthcare application and/or the residential facility where the resident resides, and the like.

The illustrated interface 200 includes a portion 215 providing demographics or details of the resident. Specifically, in the illustrated example, the portion 215 includes the resident’s name (“Betty Johnson”), a picture of the resident, the assigned unit, room, and/or bed of the resident (e.g., in the “North” building, room 102, bed “A”), the resident’s current status (e.g., “Resident,” as compared to if the patient was temporarily hospitalized or otherwise out of the facility), as well as the resident’s birthday (e.g., February 2, 1945).

In the illustrated example, the portion 215 also includes an icon 220 that the user may use or select (e.g., touching or clicking on the icon 220) to view additional information about the resident. For example, clicking the icon 220 may open a more detailed resident profile including information such as the resident’s primary physician, mailing address, email address, phone number, contact information for the resident and/or trusted individuals of the resident, and the like.

In the depicted example, the interface 200 also includes a portion 225 providing heath updates for the resident. Generally, the portion 225 may indicate the number of updates (e.g., the number of new records or events that have been recorded since the user last opened the resident’s profile), the type(s) of updates, and the like. For example, in the illustrated portion 225, the icons 230 and 235 indicate that a total of eight health updates are available for review. In some embodiments, the different icons 230 and 235 may be used to indicate different types of updates (e.g., where the icon 230 with stippling or color may indicate seven medication updates, while the icon 235 without stippling may indicate one diagnosis update). In some embodiments, the portion 225 may include various icons, each having various shapes, sizes, colors, or other designs to indicate the updates (e.g., to highlight the number of updates that are new to the user, to highlight the more important updates, and the like).

In the illustrated example, the portion 225 includes an icon 240 (labeled “More”) which the user may select to view more information about the health updates. For example, in some embodiments, the user may select the icon 240 to view an activity timeline of the health updates for the patient, as discussed in more detail below.

The illustrated example also includes a portion 245 containing information related to chats or conversations between. For example, in the illustrated interface 200, the user has a first conversation 250A related to Nursing at the “Meadowbrook” residential facility with an individual named Stuart Cooper and three others (not named). The most recent message in this conversation 250A was received today. Further, the user has a conversation 250B with Doctor Jamie Madison of Meadowbrook, where the last message was received yesterday. In some embodiments, the user can select any of the conversations 250 to re-open the chat, allowing the user to read any received messages and/or send a new message to the group. In some embodiments, the portion 245 includes a picture for each conversation 250 (e.g., depicting the person or persons with whom the user is speaking).

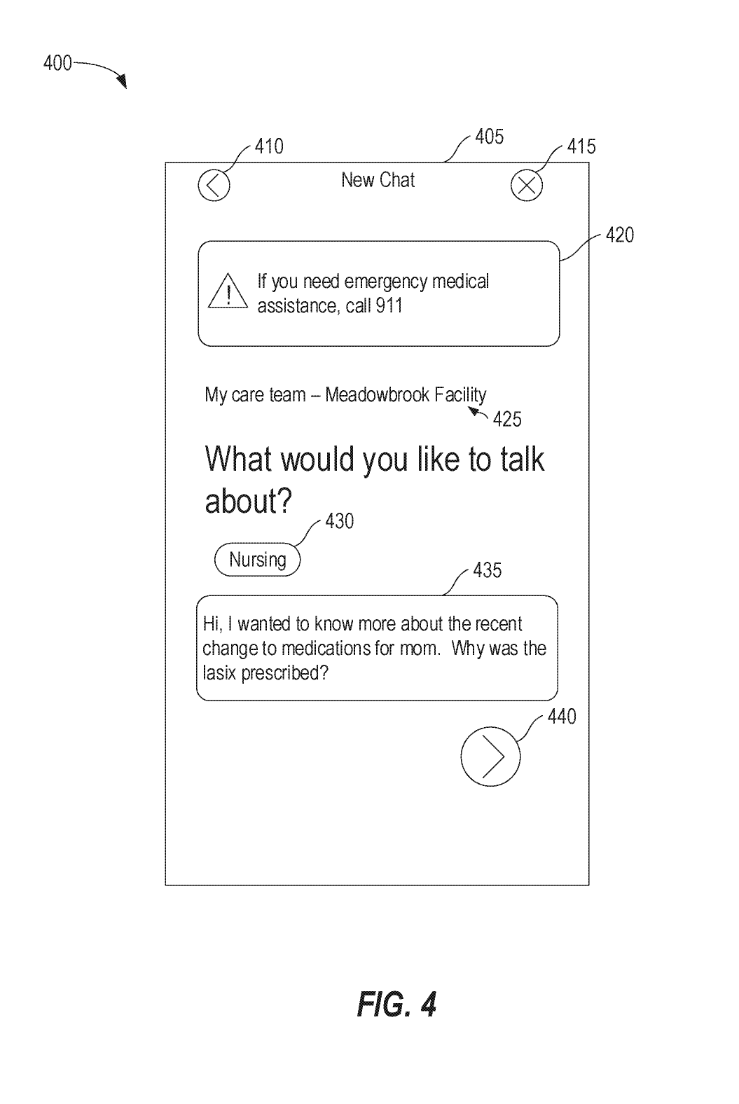

In the illustrated example, the portion 245 also includes an icon 255 (labeled “New Chat”) that allows the user to initiate a new conversation with one or more individuals. One example for initiating a new conversation is discussed in more detail below with reference to FIG. 4.

Advantageously, the interface 200 may be automatically constructed and populated (e.g., by the application system 105 of FIG. 1) to provide the user with relevant information in a rapid and efficient manner. For example, when the user authenticates and/or selects the particular resident, the healthcare application may automatically retrieve the relevant information for the resident, updating the interface 200 for effective communication.

### Example Interface Depicting a Unified Activity Timeline from Disparate Record Sources

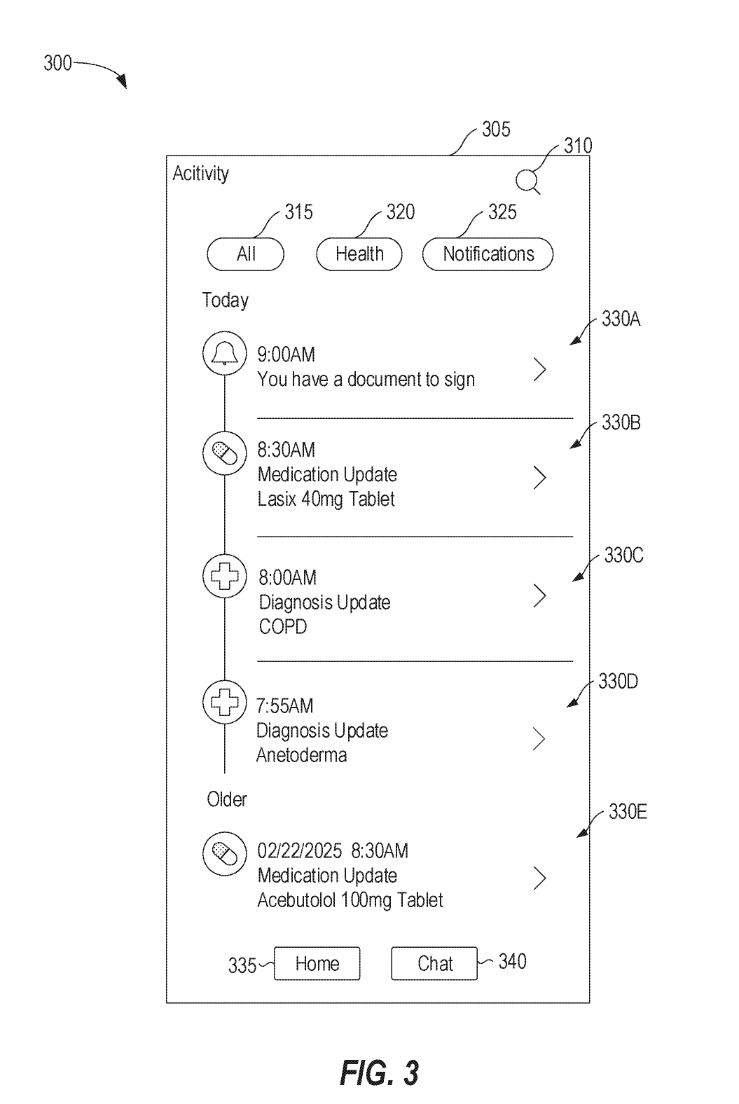

FIG. 3 depicts an example interface 300 depicting a unified activity timeline from disparate record sources, according to some embodiments of the present disclosure. In some embodiments, the interface 300 is a GUI of a healthcare application, such as the healthcare application 120 of FIG. 1. For example, the illustrated interface 300 may correspond to the healthcare application 120A executing on a client device 115, each of FIG. 1, where the contents of the interface 300 are generated or facilitated by an application system, such as the application system 105 of FIG. 1.

In the illustrated example, the interface 300 includes a portion 305 depicting an activity timeline of the resident. In some embodiments, the interface 300 is generated and output (e.g., via a GUI of the user’s device) in response to the user clicking or selecting an option to request more detail on the resident’s health. For example, the interface 300 may be generated when the user clicks the icon 240 of FIG. 2 (e.g., the “more” button on the health update section of the interface 200).

In some embodiments, the application system may generate the activity timeline by retrieving electronic health records (e.g., from the EHR 110 of FIG. 1) associated with the resident, and identifying or extracting events for the timeline. For example, the application system may extract events related to diagnoses, medication prescriptions, and the like. The application system can then generate a GUI ordering these events in time, allowing users to rapidly review the healthcare updates.

In the illustrated example, the interface 300 includes an icon 310 that the user may use (e.g., select or click) to search for information about the resident or facility. In some embodiments, the icon 310 allows the user to search the portion 305 (e.g., the activity timeline). For example, the user may search or filter the entries based on the type of the event, the name of the event, the name of the associated caregiver, and the like. For example, the user may use the icon 310 to search for a given medicine, such as to see whether (and when) the user has been prescribed it before.

In some embodiments, when the interface 300 is generated, the application system may retrieve a subset of records to populate the activity timeline. For example, rather than retrieve all available records (which may consume substantial bandwidth and cause significant memory usage on the user’s device), the application system may selectively retrieve a subset of the relevant data. For example, the application system may retrieve events within a defined period of time (e.g., the last week), or events that are “new” (e.g., recorded since the last time the user opened the activity timeline). In some embodiments, the application system may select a defined number of the most recent events (e.g., the twenty most recent events) to populate the activity timeline. Advantageously, this selective record retrieval may reduce the latency and computational expense of generating and providing the activity timeline while still ensuring that the most relevant information is presented first. Further, in some embodiments, when a user searches for more updates (e.g., via the icon 310), the application system may search the already-retrieved updates first (prior to searching the EHR repositories (or other sources)) in order to minimize the computational expense of the search. In some embodiments, the user may request additional events, such as by scrolling to the bottom of the activity timeline, searching for events not depicted on the timeline, and the like.

In the illustrated interface 300, the portion 305 also includes filter options 315, 320, and 325. For example, by selecting the option 315, filter(s) of the activity timeline may be removed to show all events or updates (regardless of type). Similarly by selecting the option 320, the activity timeline can be filtered to only include updates or events related to “health” of the resident (e.g., new diagnoses, updates regarding progression or recovery from various conditions, and the like). Further, by selecting the option 325, the activity timeline can be filtered to only include updates related to “notifications” (e.g., events that require input from the user), such as requests for the user to review and sign documents. Although three filter options 315, 320, and 325 are depicted for conceptual clarity, in some embodiments, the interface 300 may include any number and variety of filter options.

In the illustrated example, the activity timeline includes an ordered sequence of events 330A-E (e.g., ordered by date and time in descending order, such that the most recent event is at the top of the list). In some embodiments, as discussed above, each event 330 is generated by extracting data from one or more corresponding medical records of the resident. For example, one or more natural language processing (NLP) techniques may be used to evaluate EHR (e.g., written notes) to identify predefined events such as diagnoses, condition updates, prescribing (or adjust prescriptions of) medication, and the like. When such an event is identified, the application system (or another system) may then extract relevant information depending on the particular type of event. For example, for a “diagnosis” type event, the application system may extract details such as the diagnosing clinician, the disorder that was diagnosed, and the like. Similarly, for a “medication” type event, the application system may extract details such as the prescribing physician, the formula and/or brand name of the medication, the dosage, the format (e.g., liquid, injection, tablet, and the like), and the like.

In some embodiments, in addition to or instead of using rules-based and/or NLP-based techniques, the application system may use machine learning, such as language models or large language models (LLMs) to summarize the various reocrds in order to generate or supplement the events 330. Advantageously, this allows the application system to dynamically generate health update events automatically based on existing clinical records, allowing users to quickly view the most important or relevant information without requiring the user to manually review the EHR.

As illustrated, the activity timeline in the depicted interface 300 includes an event 330A of a “notification” type. The event 330A includes an icon depicting or indicating the type of the event. Specifically in the illustrated example, the icon is a bell to indicate that the user’s input or attention is needed. As illustrated, the event 330A also specifies the time of the event (e.g., the time when the medical record, from which the event was generated, was recorded or updated) (e.g., 9:00am) and includes a brief summary of the event (e.g., indicating that there is a document for the user to review and sign).

In some embodiments, the user may click or select the event 330A (e.g., using the icon on the right side of the interface 300) to view more information about the event. For example, in some embodiments, selecting this icon may cause the application system to retrieve and provide the records themselves (or links to the records) used to generate the event 330A, or to view additional extracted information such as the individual that requested the user’s signature. In some embodiments, selecting the icon may cause the application system to perform different actions depending on the event type. For example, in the case of a notification event (e.g., a request to sign a document), the icon may cause the application system to retrieve and output the document, allowing the user to review and sign without leaving the healthcare application.

In the illustrated example, the activity timeline in the depicted interface 300 further includes an event 330B of a “medication update” type. The event 330B includes an icon depicting or indicating the type of the event. Specifically in the illustrated example, the icon is a medication (e.g., a pill) to indicate that the event relates to medication. As illustrated, the event 330B also specifies the time of the event (e.g., the time when the medication was prescribed) (e.g., 8:30am) and further includes the name, chemical formula, or other identifying information for the medication. In the depicted example, the user was prescribed 40mg tablets of Lasix.

In some embodiments, as discussed above, the user may click or select the event 330B to view additional information, such as to identify the prescribing healthcare professional, to request information about the user’s other medication(s), and the like.

As another example of medication events, the activity timeline includes an event 330E of the “medication update” type. The event 330E includes an icon depicting or indicating the type of event, and also specifies the time of the event (e.g., the time when the medication was prescribed) (e.g., 8:30am on February 22, 2025) and further includes the name, chemical formula, or other identifying information for the medication. In the depicted example, the user was prescribed 100mg tablets of Acebutolol. In some embodiments, as discussed above, the user may click or select the event 330E to view additional information, such as to identify the prescribing healthcare professional, to request information about the user’s other medication(s), and the like.

In the illustrated example, the activity timeline in the depicted interface 300 further includes two events 330C and 330D of a “diagnosis update” type. The events 330C and 330D further include an icon depicting or indicating the type of the event. Specifically in the illustrated example, the icon is a cross symbol to indicate that the event relates to health of the resident. As illustrated, the events 330C and 330D also specify the time of each event (e.g., the time when the diagnosis was made) (e.g., 8:00 and 7:55am, respectfully) and further includes the name of the disorder. In the depicted example, the event 330C corresponds to a diagnosis of chronic obstructive pulmonary disease (COPD), while the event 330D corresponds to a diagnosis of anetoderma.

In some embodiments, as discussed above, the user may click or select the events 330C and/or 330D to view additional information, such as to identify the diagnosing healthcare professional, to request information about the user’s other medication(s), and the like.

In the illustrated example, the interface 300 further includes two icons near the bottom of the screen: a first icon 335 labeled “home” to exit the activity timeline (e.g., to return the first interface 200 of FIG. 2) and a second icon 340 labeled “chat” (e.g., to launch a new chat or communication). For example, in some aspects, the icon 340 may serve the same purpose as icon 255 of FIG. 2. In some embodiments, when the user uses the icon 340 to initiate a chat, the application system may prompt the user to select which event 330, if any, the chat is related to. This can facilitate or streamline the chat process.

### Example Interface to Provide Secure Chat Functionality

FIG. 4 depicts an example interface 400 to provide secure chat functionality, according to some embodiments of the present disclosure. In some embodiments, the interface 400 is a GUI of a healthcare application, such as the healthcare application 120 of FIG. 1. For example, the illustrated interface 400 may correspond to the healthcare application 120A executing on a client device 115, each of FIG. 1, where the contents of the interface 400 are generated or facilitated by an application system, such as the application system 105 of FIG. 1.

In the illustrated example, the interface 400 includes a portion 405 to initiate a new chat or conversation between the user and one or more other individuals (e.g., with a resident, with one or more caregivers, with social workers, and the like). In some embodiments, the interface 400 is generated and output (e.g., via a GUI of the user’s device) in response to the user clicking or selecting an option to initiate a chat. For example, the interface 400 may be generated when the user clicks the icon 255 of FIG. 2 (e.g., the “New Chat” button on the interface 200) and/or the icon 340 of FIG. 3 (e.g., the “chat” icon on the activity timeline of the interface 300).

In the illustrated example, the interface 400 includes an icon 410 to return to a previous screen or interface (e.g., to return to the overall chat list or the activity timeline) as well as an icon 415 to close the chat entirely (e.g., to return to the home screen or patient details interface). Additionally, in the illustrated example, the interface 400 includes a portion 420 to note that the chat functionality of the healthcare application is not intended to replace emergency services. Specifically, the portion 420 notes that the user should call 911 (or some other emergency line) if they are in need of emergency assistance.

At 425, the interface 400 specifies which residential facility (or “care team” is relevant to the chat or resident. Specifically, the illustrated example notes that the chat is related to the “Meadowbrook Facility” care team (e.g., the chat will either be with a staff member of that facility, or with another individual to discuss the Meadowbrook facility). In some embodiments, this context is automatically populated by the application system (e.g., based on the facility in which the resident resides). For example, when the user initiates a new chat from the profile of a given resident, the application system may infer or assume that the user wishes to discuss that resident and/or the facility in which that resident resides. In some embodiments, the user may click or select the portion 425 to change the context, if desired (e.g., to discuss or talk with a different care team, such as hospital staff where the resident is currently being treated).

As illustrated, the interface 400 then asks what the user would like to discuss, and provides a portion 430 where the user can select the context or target of their inquiry. For example, in the illustrated interface 400, the portion 430 notes that the request or conversation relates to “Nursing” in the Meadowbrook facility. In some embodiments, the portion 430 may be automatically populated by the application system. For example, if the user initiates a chat from the activity timeline and indicates a particular event as the topic of discussion, the application system may populate the portion 430 with the corresponding context. As one example, if the user indicates that they want to discuss a recent diagnosis, the application system may update the portion 430 to indicate that the context is medical updates, diagnoses, physician discussion, or the like. As another example, if the user indicates that they want to discuss a recent invoice or other document in the timeline, the portion 430 may be updated to indicate that the user wishes to talk with the billing or financial assistance staff.

In some embodiments, the application system may select or interact with the portion 430 to indicate the desired topic (e.g., by selecting the topic from a dropdown list, or by typing the topic in). In some embodiments, the portion 430 may be automatically inferred or generated based on the content of the question. For example, after the user enters a textual question, the healthcare application may use one or more NLP techniques to infer to determine the appropriate context (e.g., whether the question should be provided to a nurse, a doctor, a social worker, and the like).

In the depicted interface 400, the user may then use the portion 435 to enter natural language (e.g., unstructured) input, such as text, indicating their inquiry or question. In the illustrated example, the user would like to know why the resident (the user’s mother) was recently prescribed Lasix medication. In some embodiments, the user can manually type their inquiry into the portion 435. In some embodiments, the user may dictate their inquiry (e.g., by recording a spoken question, allowing the healthcare application to generate a corresponding textual inquiry, such as by using one or more speech-to-text techniques).

When the user has completed their query, the icon 440 may be used to send the message and initiate the conversation or chat. Advantageously, the user may use the interface 400 to initiate chats within the healthcare application itself, rather than relying on external messaging solutions. This can streamline and simplify the chat process, while further ensuring that the relevant context and information is readily accessible. Further, in some embodiments, the chat system can be designed to satisfy HIPAA and other privacy or security concerns, ensuring that all communications are secure and permissible by relevant regulations or policies.

Further, in the illustrated example, the user need not select any specific user or target for their inquiry and, in some cases, need not even specify the general department or type of user to respond. For example, as discussed above, the healthcare application may infer or determine that the question relates to medication prescriptions, and may therefore determine that the question should be forwarded to a staff member who is able to respond to such inquiries.

In some embodiments, as discussed above, in addition to inferring which department should receive the inquiry, the application system may evaluate various information such as the staffing 112 of FIG. 1 to determine the specific individual who should receive the inquiry. For example, if the application system determines that the inquiry relates to the resident’s current state (e.g., “how is dad feeling this morning?”), the application system may evaluate the staffing to identify which nurse or other caregiver is currently assigned to assist the resident at the current time. The inquiry can then be forwarded to this specific caregiver. This prevents waste and confusion caused by inaccurate message targeting.

### Example Interface to Provide Secure Chat Functionality with Caregivers

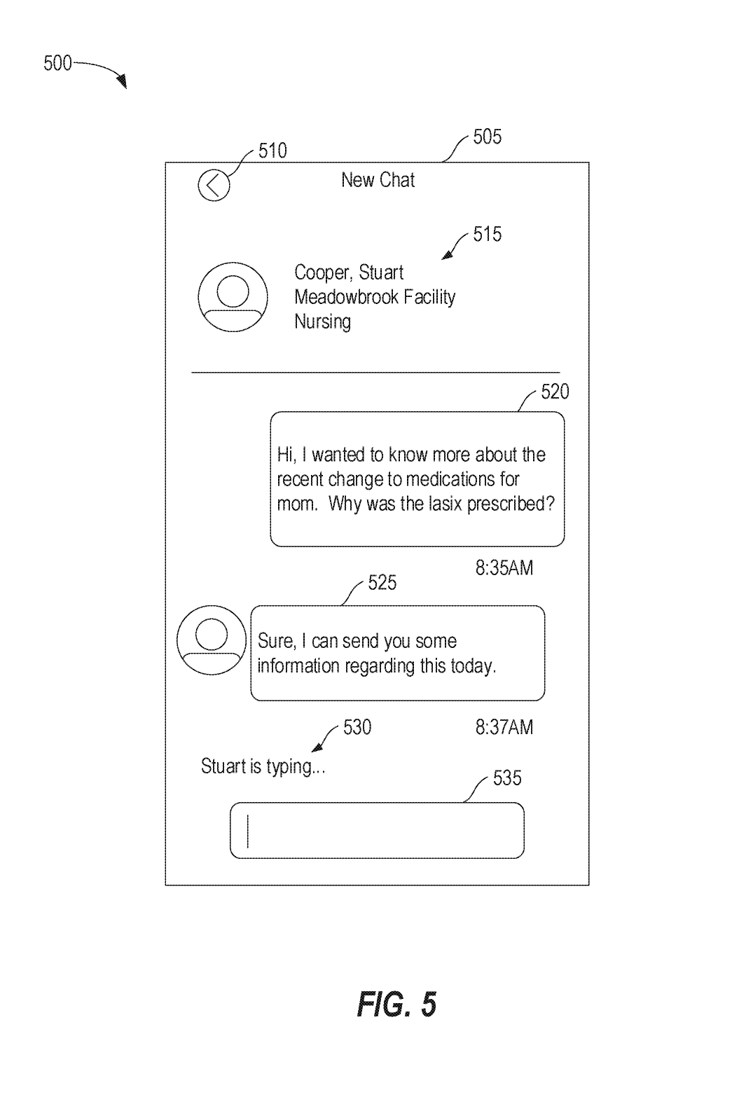

FIG. 5 depicts an example interface 500 to provide secure chat functionality with caregivers, according to some embodiments of the present disclosure. In some embodiments, the interface 500 is a GUI of a healthcare application, such as the healthcare application 120 of FIG. 1. For example, the illustrated interface 500 may correspond to the healthcare application 120A executing on a client device 115, each of FIG. 1, where the contents of the interface 500 are generated or facilitated by an application system, such as the application system 105 of FIG. 1.

In the illustrated example, the interface 500 includes a portion 505 to for a newly initiated chat or conversation between the user and one or more other individuals (e.g., with a resident, with one or more caregivers, with social workers, and the like). In some embodiments, the interface 500 is generated and output (e.g., via a GUI of the user’s device) in response to the user clicking or selecting a prior chat (e.g., selecting the conversation 250A of FIG. 2) to continue the conversation, and/or by clicking or selecting an icon to initiate a new chat (e.g., the icon 440 of FIG. 4).

In the illustrated interface 500, the user may use the icon 510 to exit the chat (e.g., to return to the previous window). Further, the portion 515 indicates the individual (or individuals) to whom the inquiry was forwarded and/or the individual (or individuals) who are participating in the conversation. Specifically, in the illustrated example, the conversation is with Stuart Cooper, who is on the nursing staff of the Meadowbrook Facility. In some embodiments, as illustrated, this portion 515 may also include an image of the individual.

In the illustrated example, the chat includes a first message sfrom the user (e.g., generated based on the user’s input on the interface 400 of FIG. 4). Specifically, the message 520 used to initiate the chat includes a request for information about why a resident was prescribed Lasix. In the illustrated example, the interface 500 also indicates the time when the message was entered or received (e.g., at 8:35AM in the illustrated example).

Further, the chat includes a new message 525 from the other individual (e.g., from Stuart Cooper), along with an image used to rapidly identify the user that transmitted the message 525. In the illustrated example, the message 525 indicates that Stuart will send more information “today” regarding the Lasix prescription. This message was sent at 8:37AM. Further, as indicated by the portion 530, Stuart is still typing a new message for the chat.

In some embodiments, as discussed above, the message 520 was forwarded to Stuart automatically (e.g., because Stuart is the current nurse assigned to the resident, or because Stuart prescribed or entered the Lasix prescription). In some embodiments, if Stuart needs more input before responding, Stuart can use a similar interface to initiate a chat with the relevant user(s). For example, Stuart may launch a new chat and request the particular doctor that prescribed the Lasix and/or the doctor on call, asking that individual to provide a response for the message 520.

In the illustrated example, 500, the interface 500 further includes a portion 535 where the user can enter additional messages (e.g., using written text or voice-to-text, as discussed above) in the chat.

Advantageously, as discussed above, this chat functionality enables seamless and automated message forwarding to the relevant individual(s) at any given time, ensuring that the user’s inquiry can be responded to quickly and accurately. Further, the user need not investigate as to who the proper recipient is. Additionally, in some embodiments, the message may be forward to the caregiver’s mobile device (e.g., the caregiver device 125 of FIG. 1) rather than a fixed terminal or other device, allowing the caregiver to rapidly see and respond to the message without confusion or delay. Moreover, as discussed above, the chat system may satisfy HIPAA and any other policy or regulatory concerns, allowing caregivers to quickly provide useful information without concerns about privacy or restrictions on such information sharing.

### Example Interface to Provide Secure Chat Functionality with Machine Learning-Based Systems

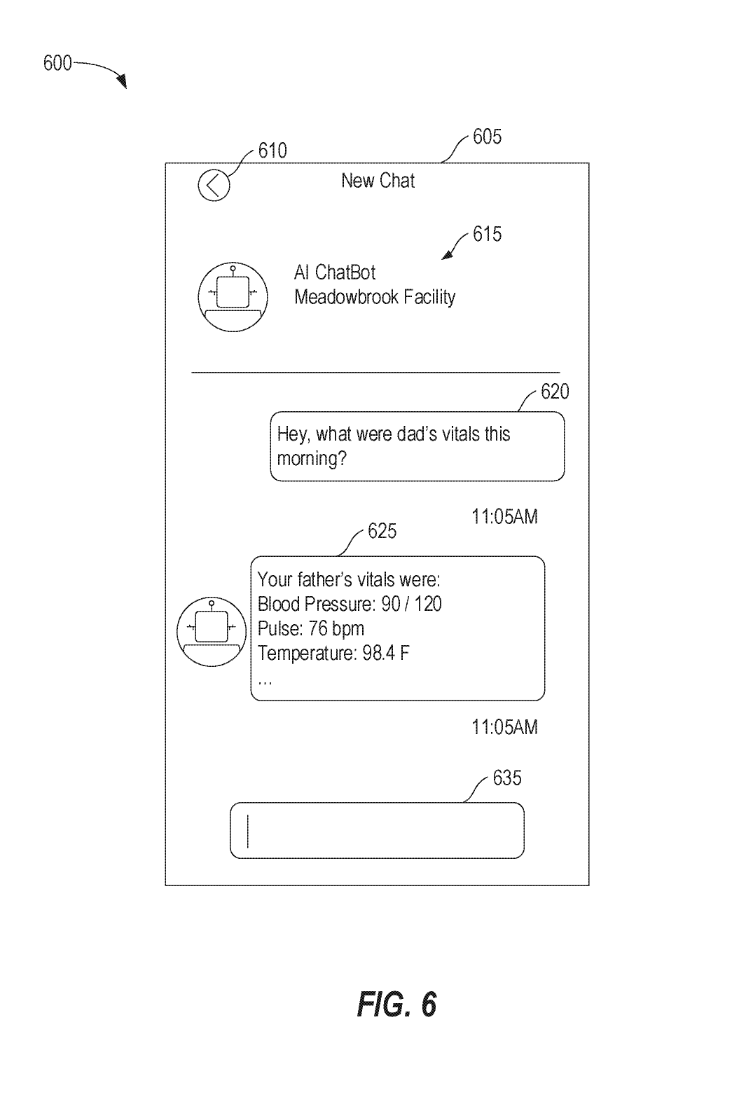

FIG. 6 depicts an example interface 600 to provide secure chat functionality with machine learning-based systems, according to some embodiments of the present disclosure. In some embodiments, the interface 600 is a GUI of a healthcare application, such as the healthcare application 120 of FIG. 1. For example, the illustrated interface 600 may correspond to the healthcare application 120A executing on a client device 115, each of FIG. 1, where the contents of the interface 600 are generated or facilitated by an application system, such as the application system 105 of FIG. 1.

In the illustrated example, the interface 600 includes a portion 605 to for a newly initiated chat or conversation between the user and a chat bot. In some embodiments, the interface 600 is generated and output (e.g., via a GUI of the user’s device) in response to the user clicking or selecting a prior chat (e.g., selecting a conversation 250 of FIG. 2) to continue the conversation, and/or by clicking or selecting an icon to initiate a new chat (e.g., the icon 440 of FIG. 4).

In the illustrated example, rather than the user inquiry being forwarded to an individual caregiver, the application system instead provides the inquiry to a chat bot. For example, in some aspects, the application system may evaluate new inquiries (e.g., the inquiries used to initiate a new chat) to determine whether the inquiry can be adequately handled using machine learning (ML) and/or artificial intelligence (AI), or whether a real individual should be contacted. In some embodiments, the application system may use defined mappings indicating what contexts or inquiries are adequately handled using AI (where all other inquiries are forwarded to human caregivers). In some embodiments, the application system may process the input inquiry using AI or ML (e.g., a lightweight classification model that uses relatively fewer computational resources, as compared to the chat bot itself) to predict whether the AI is capable of generating a satisfactory response.

For example, a question about why a particular resident was prescribed a new medication is likely to require human input, and the application system may therefore forward the inquiry to a human user (e.g., the caregiver or physician caring for the patient). In contrast, a request for discrete information may be adequately handled automatically. For example, if the user requests specific information such as “what were the resident’s vitals this morning” or “when is the current invoice due,” the application system may determine that these are clear and specific requests for information that is well-bounded and readily answered using ML. Specifically, for the former, the application system may evaluate the resident’s records from “this morning” to determine what vital(s) were recorded (e.g., blood pressure, heart rate, temperature, and the like), and may then generate an automated response including this information. Similarly, for the latter, the application system may find the current, most recent, or outstanding invoice(s) for the resident, and may determine and output a response with the due date of the invoice.

Advantageously, by using a classification mode (or other lightweight analysis, such as keyword matching or rules-based evaluations) to determine whether to use machine learning (e.g., an AI chat bot) to respond to a given query, the application system can significantly reduce the computational expense of generating responses. That is, the application system may selectively use machine learning models (e.g., LLMs), which may consume substantial computational resources to generate responses, only for a subset of user requests (rather than using such complex models to process all inputs). This selective machine learning processing based on comparatively less computationally expensive initial evaluations (e.g., using a lightweight classifier) reduces not only the computational expense of the system, but also response latency (e.g., because resources are reserved for responses that can actually use such resources), power consumption of the application system, heat generation of the system, and the like.

Stated differently, the application system may limit use of the more complex generative models to only when the initial evaluations (e.g., performed using less complex classifiers or rules-based keyword matching) reflects a need or benefit from such complex models, which avoids excess computational expense. Similarly, if the more complex models are hosted on remote systems (e.g., in the cloud), which is common for such large generative models, the selective use of such models can substantially reduce the traffic volume on the network and hindrance of network performance. That is, the application system may avoid sending such user requests to the cloud-based generative models in some cases, thereby reducing network traffic and congestion.

In the illustrated example, the user may use the icon 610 to exit the chat (e.g., to return to the previous window) as discussed above. Further, the portion 615 indicates that the request is being answered by a chat bot. Specifically, in the illustrated example, the portion 615 indicates that the chat is with an “AI ChatBot” associated with the Meadowbrook Facility, and includes a picture of a robot. Generally, the portion 615 may include any information to ensure that it is clear that the answer was generated by AI/ML (as compared to a human user). This can improve trust in the system, as the user always knows whether they are talking with a human caregiver or an automated system.

In the illustrated example, the chat includes a first message 620 from the user (e.g., generated based on the user’s input on the interface 400 of FIG. 4). Specifically, the message 620 used to initiate the chat includes a request for what the resident’s vitals were when last recorded. In the illustrated example, the interface 600 also indicates the time when the message was entered or received (e.g., at 11:05AM in the illustrated example).

In response, the message 625 is automatically generated to indicate the recorded vitals. In this way, the application system can selectively respond to inquiries using machine learning in some cases, reducing delay and burden on human users while preserving integrity and accuracy of the chat system (e.g., by only selectively using the automated responses). Although not depicted in the illustrated example, in some embodiments, the interface 600 may include a button or icon to specifically request human intervention in the chat. For example, if the user is not satisfied with the message 625 or otherwise wants a human to respond, a button may be provided to cause the application system to forward the message 620 and/or the entire chat to a human, as discussed above (e.g., selecting the proper caregiver automatically based on the context of the question).

Similarly, in the illustrated example, the user may use the portion 635 to enter another message or inquiry. In some embodiments, this further input may generally include unstructured natural language, and may include related or unrelated questions. In some embodiments, the user may use the portion 635 to request human input.

In some embodiments, if the user enters a new message via the portion 635, the application system may evaluate this new question to determine whether the new inquiry can also be answered using AI, or if the inquiry should be forwarded to a human. In some embodiments, if the follow-up request can be answered using AI, the application system may generate a new response automatically and enter this response in the chat. In some embodiments, if the application system determines that the follow-up request requires human input, the application system may forward the entire chat to an identified caregiver or other human. In some embodiments, the application system accomplishes this forwarding by adding the human to the chat. In some embodiments, when a human caregiver is added, the application system may remove the Chatbot from the chat, allowing the conversation to continue between the user and the caregiver(s).

### Example Interface for Assignment Selection

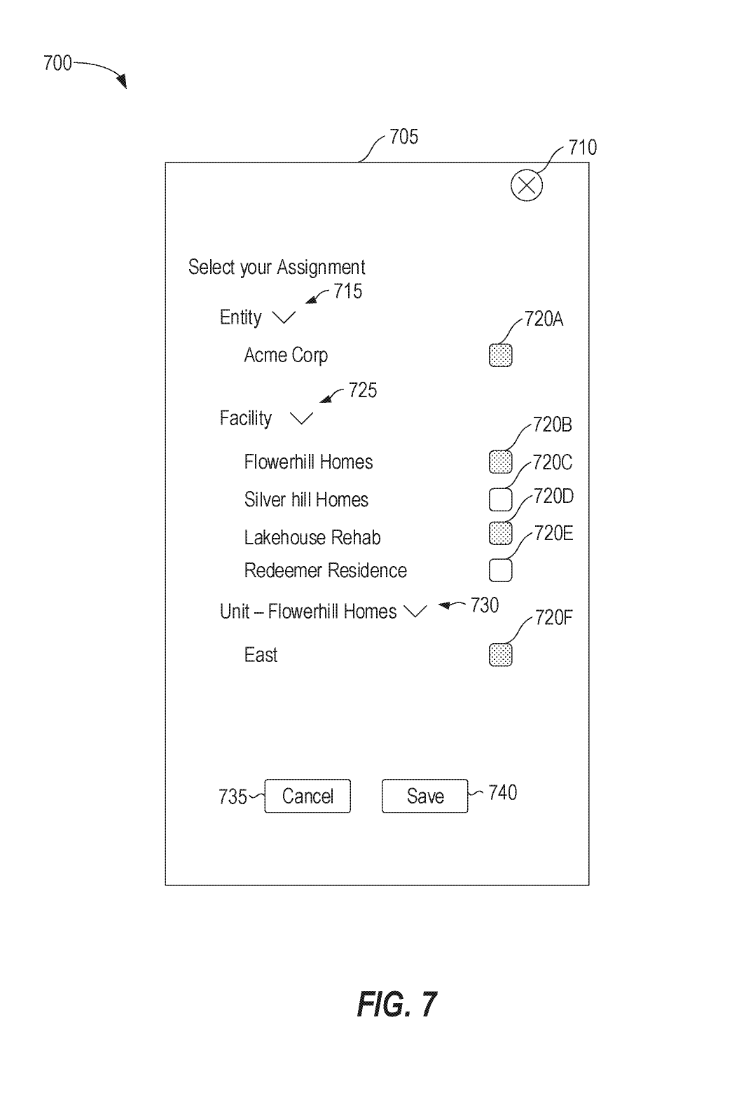

FIG. 7 depicts an example interface 700 for assignment selection, according to some embodiments of the present disclosure. In some embodiments, the interface 700 is a GUI of a healthcare application, such as the healthcare application 120 of FIG. 1. For example, the illustrated interface 700 may correspond to the healthcare application 120B executing on a caregiver device 125, each of FIG. 1, where the contents of the interface 700 are generated or facilitated by an application system, such as the application system 105 of FIG. 1.

In the illustrated example, the interface 700 is displaying an assignment screen 705 allowing the user (e.g., a caregiver) to select which residential care facility (or facilities) they are associated with or assigned to. For example, in some embodiments, upon the caregiver opening the healthcare application and/or authenticating (e.g., logging in using a username and password, using a biometric passcode such as a fingerprint or face scan, and the like), the healthcare application may identify any facilities associated with the caregiver, or may ask the caregiver to clarify or confirm their current assignment.

For example, at portion 715, the caregiver may select which entity or entities they are working for or with (e.g., for the current shift). In the specific example, as indicated by the stippling in the block 720A, the caregiver has indicated that they are working in association with “Acme Corp” at the moment. In some embodiments, the portion 715 may be populated with a list of alternative healthcare entities that use the application system, allowing users to select which they are working with.

Further, as illustrated in the portion 725, the interface 700 asks the caregiver to indicate which particular facility or facilities they are working in (either for the current shift, or in general). In particular, as illustrated, the caregiver indicated (via the blocks 720B and 720D) that they are working in the Flowerhill Homes facility and the Lakehouse Rehab facility. As indicated by the lack of stippling in the blocks 720C and 720E, the user is not (currently) assigned to work in Silver hill homes or Redeemer Residences.

Further, at portion 730, the interface 700 may ask the caregiver to indicate specific unit(s) (e.g., buildings) where applicable. For example, as indicated by the stippling 720F, the caregiver has indicated that they are assigned to work in the East building of the Flowerhill Homes facility.

In some embodiments, some or all of the interface 700 may be prepopulated (e.g., automatically by the application system) based on staffing information. For example, when the user authenticates or logs in, the application system may evaluate the staffing 112 of FIG. 1 to identify the relevant entities, facilities, and/or units to which the user is assigned.

In the depicted example, the assignment screen 705 includes an icon 710 allowing the user to exit or quit the assignment process, if desired. The interface further includes an icon 735 to cancel the assignment (e.g., to undo any changes made), as well as an icon 740 to save the updated assignment (e.g., to enter or memorialize the changes made).

### Example Interface for Resident Selection

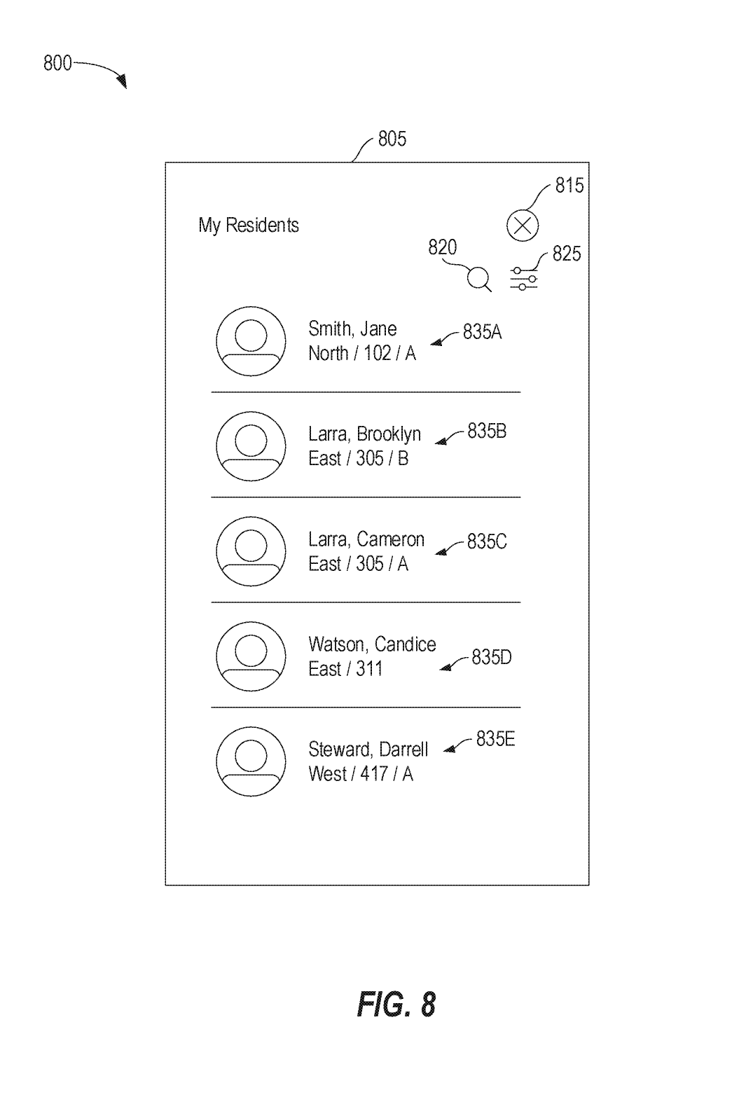

FIG. 8 depicts an example interface 800 for resident selection, according to some embodiments of the present disclosure. In some embodiments, the interface 800 is a GUI of a healthcare application, such as the healthcare application 120 of FIG. 1. For example, the illustrated interface 800 may correspond to the healthcare application 120B executing on a caregiver device 125, each of FIG. 1, where the contents of the interface 800 are generated or facilitated by an application system, such as the application system 105 of FIG. 1.

In the illustrated example, the interface 800 includes a portion 805 indicating the residents that are currently assigned to the caregiver (e.g., determined based on the staffing information and/or based on the assignment selections indicated above on the interface 700 of FIG. 7). In the illustrated example, the portion 805 includes an icon 815 to close the resident information (e.g., to return to a caregiver home screen of the application).

Further, in the illustrated example, the interface includes an icon 820 to search for particular residents or facility information. For example, the user may search based on details like the resident’s name, the facility, unit, and/or room in which the resident resides, and the like. In the illustrated example, the interface 800 also includes an icon 825 to allow the user to adjust configurations, settings, filters, and the like. For example, the user may filter which facilities are visible.

In the illustrated example, each resident is depicted as an entry 835A-E with information including the resident’s name, a picture of the resident, the location of the resident, and the like. Specifically, in the illustrated interface 800, the entry 835A corresponds to a resident named Jane Smith who lives in the North building, room 102, bed A. Further, as indicated by entries 835B and 835C, the residents Brooklyn and Cameron Larra live in the East building, room 305, beds B and A, respectively. Additionally, as illustrated by the entry 835D, a resident Candice Watson lives in room 311 of the East building, and as illustrated by the entry 835E, the resident Darrel Steward lives in room 417 of the West building.

Advantageously, using the interface 800, the user can quickly review which resident(s) they are assigned to at any given time. In some embodiments, the user may select any of the residents (e.g., by clicking or selecting the corresponding entry 835) to view more information about the resident. For example, in some embodiments, selecting one of the entries 835 may cause the healthcare application to open the interface 200 of FIG. 2 having details about the particular selected resident.

### Example Interface for Evaluating Synchronized Data across Residents

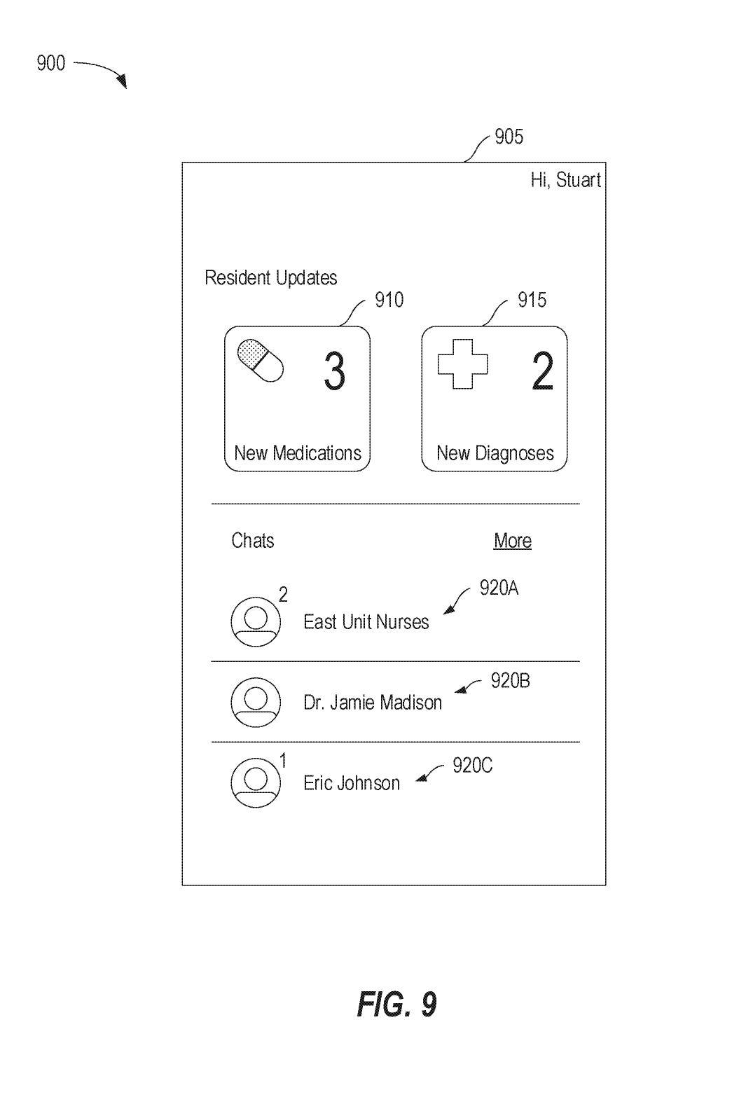

FIG. 9 depicts an example interface 900 for evaluating synchronized data across residents, according to some embodiments of the present disclosure. In some embodiments, the interface 900 is a GUI of a healthcare application, such as the healthcare application 120 of FIG. 1. For example, the illustrated interface 900 may correspond to the healthcare application 120B executing on a caregiver device 125, each of FIG. 1, where the contents of the interface 900 are generated or facilitated by an application system, such as the application system 105 of FIG. 1.

In the illustrated example, the interface 900 includes a portion 905 that acts as a home screen or main page for a caregiver. Specifically, as illustrated, the portion 905 includes a brief greeting of the user (e.g., “Hi Stuart”) to identify the caregiver, as well as a set of resident updates. In the illustrated example, the resident updates can generally include a summary of any new events or information relating to the resident(s) to whom the caregiver is assigned. For example, the resident updates may be automatically updated to illustrate any new medications or diagnoses for the resident(s) that the user has not yet seen (e.g., because the update was entered after the user last viewed the interface 900).

In the illustrated example, the residents associated with the caregiver have three new medication updates (as indicated by the icon 910) and two new diagnoses (as indicated by the icon 915). In some embodiments, the caregiver may select or click either icon 910 or 915 to view more information. For example, in some embodiments, selecting the icon 910 may cause the application system to generate an output a list of the resident(s) that have the new medications, as well as information about what the new medications are. In some embodiments, this list may be presented in a similar manner to the activity timeline of FIG. 3, where the application system can quickly review the relative timing of the prescriptions, as well as the details of each prescription (e.g., who prescribed it, what the medicine is, what the dosage is, and the like).

Further, as illustrated in the portion 905, the interface 900 can include a set of ongoing or active chats or conversations that include the caregiver. Specifically, in the illustrated example, the caregiver is part of a first conversation 920A for “East Unit Nurses,” such as to discuss any unit-wide topics of relevance. In some embodiments, in addition to the image depicting the user (or users) in the conversation 920A, the depicted entry includes a number (e.g., “2”) indicating the number of new messages in the chat that the caregiver has not yet seen.

The illustrated example further includes a chat 920B with Dr. Jamie Madison (e.g., the doctor that prescribed medicine for one of the caregiver’s residents) as well as a chat 920C with Eric Johnson (e.g., a family member of one or the caregiver’s residents). For example, as discussed above, a user (e.g., Eric Johnson) may create an inquiry asking why their mother was prescribed a given medication. This inquiry may be automatically forwarded to the caregiver “Stuart” (via the interface 900) based on determining that Stuart is the current nurse assisting Eric’s mother. Further, as discussed above, the caregiver can readily generate a message to the prescribing physician (e.g., Dr. Madison) to get more information prior to responding to the user.

In these ways, the caregiver can use the healthcare application as an all-in-one solution to merge the various data sources and communication channels in a unified and efficient manner, as compared to conventional solutions.

### Example Method for Synchronizing Data and Providing Cohesive Updates

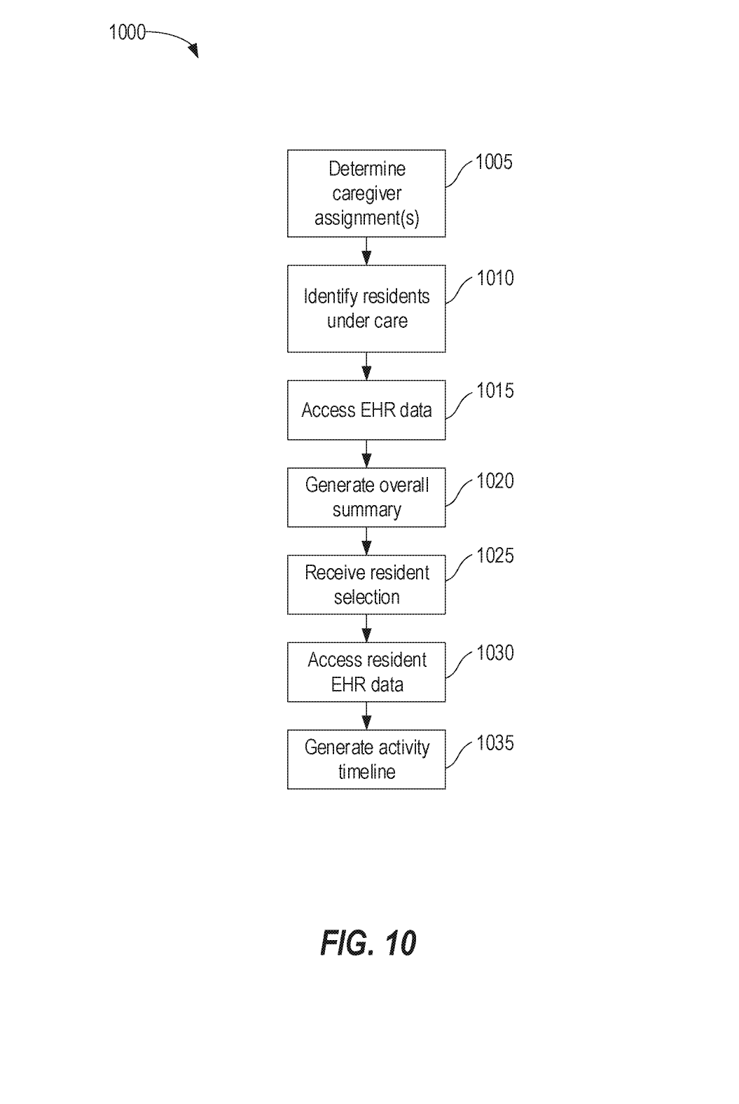

FIG. 10 is a flow diagram depicting an example method 1000 for synchronizing data and providing cohesive updates, according to some embodiments of the present disclosure. In some embodiments, the method 1000 is performed by an application system, such as the application system 105 of FIG. 1. For example, some or all of the method 1000 may be performed or implemented using a healthcare application, such as the healthcare applications 120 of FIG. 1, and/or using one or more GUIs, such as the interfaces discussed above with reference to FIGS. 2-9.

At block 1005, the application system determines a set of caregiver assignments. For example, the application system may identify which resident(s) are assigned to each caregiver at a given time, such as using the staffing 112 of FIG. 1, the interface 700 of FIG. 7, and/or the interface 800 of FIG. 8.

At block 1010, the application system identifies the set of residents under the care of the current user of a healthcare application. For example, when a caregiver authenticates or opens the healthcare application, the application system may identify the set of resident(s) to whom the caregiver is assigned during the current shift.

At block 1015, the application system accesses EHR data (e.g., the EHR 110 of FIG. 1) for the identified residents (identified at block 1010). For example, as discussed above, the application system may retrieve any new records (e.g., records from the last day, the last shift, or added since the last time the user logged into the application).

At block 1020, the application system optionally generates an overall summary for the resident(s). For example, as discussed above, if the user is a caregiver, the application system may generate a summary such as discussed above with reference to the interface 900 of FIG. 9, allowing the caregiver to quickly review what change(s) have occurred for the residents under their care.

At block 1025, the application system receives a selection of a resident with whom the user is associated. For example as discussed above, the user may manually select a resident (if they are assigned to or associated with multiple such residents), or if the user is associated with a single resident, the application system may use the user’s login or authentication as a selection of this resident.

At block 1030, the application system accesses the resident’s specific EHR data (e.g., from the EHR 110 of FIG. 1). In some embodiments, as discussed above, the application system may access the data for a given window of time (e.g., the past week), or may identify any records that were updated since the last time the user reviewed the resident data.

At block 1035, the application system generates an activity timeline for the resident, as discussed above. For example, as discussed above with reference to FIG. 3, the application system may identify records relating to defined “events” such as new diagnoses, medication changes, and the like. The application system may then generate a timeline indicating this sequence of events, with relevant data for each event automatically extracted from the corresponding record(s). The activity timeline can then be output for display via the healthcare application, as discussed above.

As discussed above, this automated process can allow users to rapidly review relevant updates and events for any given user without relying on manual search or evaluation of the healthcare records. This can significantly increase the efficiency of the data access (e.g., reducing or eliminating the wasted time and computational resources incurred by manual searching), as compared to conventional solutions.

### Example Method for Enabling User Interaction with Data Systems

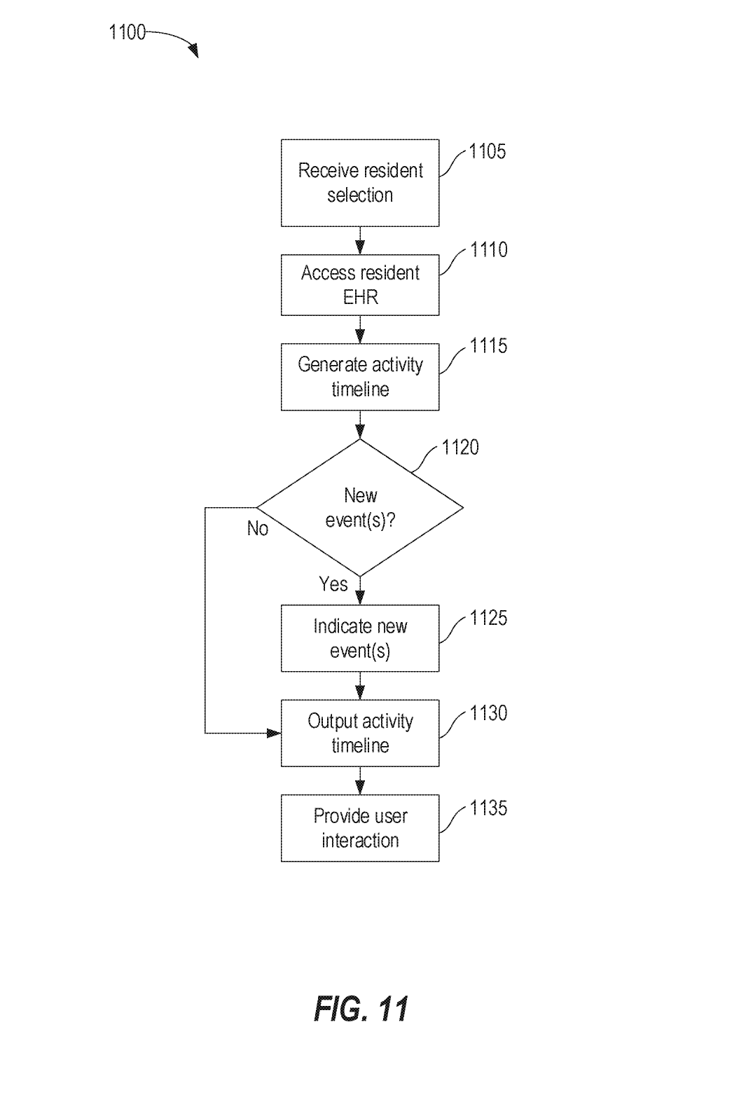

FIG. 11 is a flow diagram depicting an example method 1100 for enabling user interaction with data systems, according to some embodiments of the present disclosure. In some embodiments, the method 1100 is performed by an application system, such as the application system 105 of FIG. 1. For example, some or all of the method 1100 may be performed or implemented using a healthcare application, such as the healthcare applications 120 of FIG. 1, and/or using one or more GUIs, such as the interfaces discussed above with reference to FIGS. 2-9.

At block 1105, the application system receives a resident selection. That is, the application system receives a selection, by a user (e.g., a friend or family member) of a particular resident in a residential care facility. In some embodiments, as discussed above, if the user is linked to multiple residents, the application system may allow the user to specify which resident they wish to review. In some embodiments, if the user is associated with a single resident, the application system may interpret the user’s login or authentication as a selection of this resident.

At block 1110, the application system accesses the resident’s specific EHR data (e.g., from the EHR 110 of FIG. 1). In some embodiments, as discussed above, the application system may access the data for a given window of time (e.g., the past week), or may identify any records that were updated since the last time the user logged into the application or reviewed the resident data.

At block 1115, the application system generates an activity timeline for the resident, as discussed above. For example, as discussed above with reference to FIG. 3, the application system may identify records relating to defined “events” such as new diagnoses, medication changes, and the like. The application system may then generate a timeline indicating this sequence of events, with relevant data for each event automatically extracted from the corresponding record(s). The activity timeline can then be output for display via the healthcare application, as discussed above.

As discussed above, this automated process can allow users (e.g., friends and family members) to rapidly review relevant updates and events for any given user without relying on manual search or evaluation of the healthcare records, and without requesting explicit updates from the resident’s caregivers. This can significantly increase the efficiency of the data access (e.g., reducing or eliminating the wasted time and computational resources incurred by manual searching), as compared to conventional solutions.

At block 1120, the application system determines whether any new events have occurred in the resident’s data. For example, the application system may determine whether the resident’s EHR indicate any new diagnoses, vitals, clinician notes, observations, lab reports, medications, and the like. If no such new events are detected, the method 1100 continues to block 1130, where the application system (continues to) output the timeline for the user.

If at least one new event is detected, the method 1100 continues to block 1125, where the application system generates indication(s) of the new event(s). In some embodiments, these indications may include adding the new event(s) to the activity timeline. In some embodiments, the indications may include generating notifications or alerts for the user. In some embodiments, the application system may determine whether to generate a push alert based at least in part on the type of the new event (e.g., generating a notification when the new event is sufficiently important, or satisfies criteria specified by the user for affirmative notification). In some embodiments, the system will also ask for acknowledgment by the end user that they have received and viewed the notification. Such notification and/or the acknowledgement may then be stored clinician notes or other relevant repositories. At block 1130, the application system outputs the activity timeline (with new events added, if any) via the healthcare application (e.g., via a GUI on the user’s mobile device).

At block 1135, the application system can then generally provide or facilitate user interactions, as discussed above. For example, the application system may allow the user to request additional information about the various events, may allow the user to initiate or continue a conversation with a caregiver, and the like.

Advantageously, by organizing and providing the relevant information and functionality via a single unified application, the application system is able to significantly improve the user experience and reduce (or eliminate) computational waste and inefficiencies, as discussed above.

### Example Method for Secure Document Management

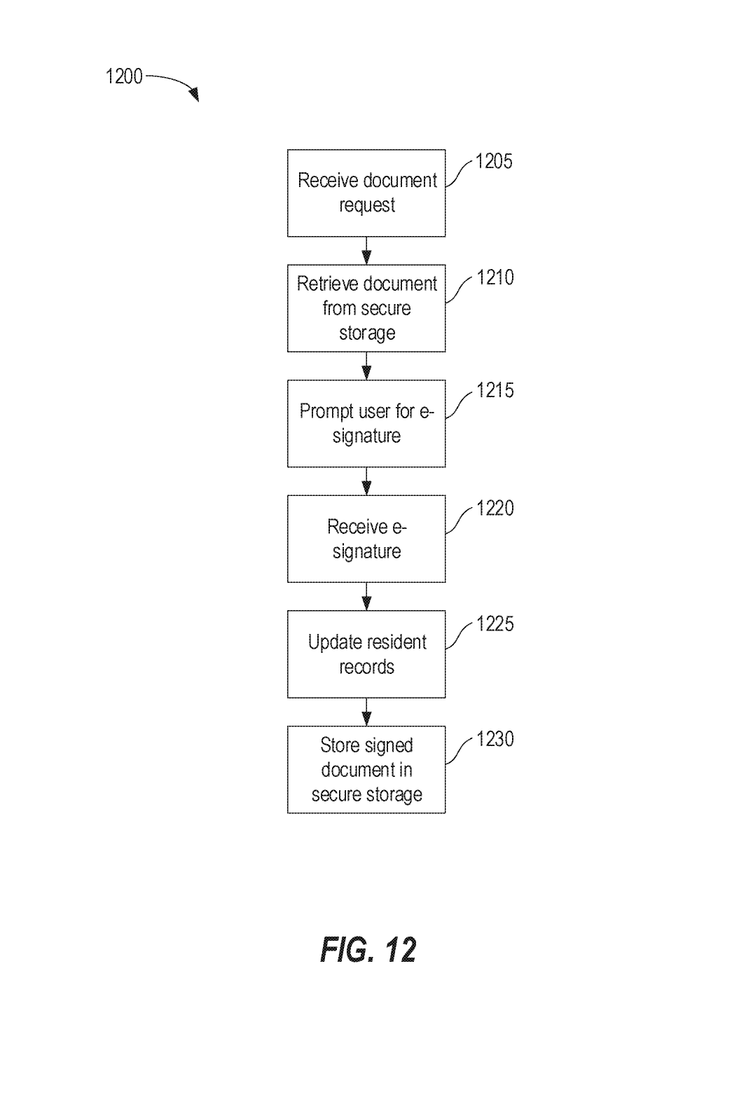

FIG. 12 is a flow diagram depicting an example method 1200 for secure document management, according to some embodiments of the present disclosure. In some embodiments, the method 1200 is performed by an application system, such as the application system 105 of FIG. 1. For example, some or all of the method 1200 may be performed or implemented using a healthcare application, such as the healthcare applications 120 of FIG. 1, and/or using one or more GUIs, such as the interfaces discussed above with reference to FIGS. 2-9.

At block 1205, the application system receives a document request. For example, as discussed above, the application system may generate notifications or events (e.g., on an activity timeline) indicating that the user should review one or more documents (e.g., consent forms, updates, and the like). In some embodiments, at block 1205, the user selects or engages with one such notification, requesting the document be provided for their review.

At block 1210, the application system retrieves the requested document from a secure document storage repository (e.g., the documents 111 of FIG. 1). In some embodiments, as discussed above, the application system may retrieve the document in response to authenticating the user and/or document request. For example, the application system may confirm that the user is listed as an individual who should have access to the document (e.g., in a security policy associated with the document and/or the storage system), that the document is still valid or relevant, and the like.

At block 1215, the application system prompts the user for an electronic signature on the document. For example, as discussed above, the application system may output the document to the user (e.g., via a GUI) to allow the user to read the document. The application system may then use an electronic signature technique to request that the user securely sign the document, indicating that they have read, understood, and/or consented to the contents.

At block 1220, the application system receives the electronic signature from the user for the document. At block 1225, the application system then updates one or more resident records (e.g., in the EHR 110 of FIG. 1) to indicate that the user has reviewed and signed the document. For example, the application system may update the activity timeline of the resident to indicate that the document has been reviewed and signed.

At block 1230, the application system stores the signed document back to the secure storage repository. In some embodiments, as discussed above, the application system may store this signed document in accordance with various regulations and/or policies (e.g., laws dictating how long medical records must be kept before disposal).

In this way, as discussed above, the application system may use an integrated signature process to provide documents for review and secure user signatures all within the healthcare application, rather than relying on external programs or systems. This approach can improve security substantially, as well as reducing latency and computational inefficiencies caused by process duplication (e.g., re-authenticating of the user, re-provisioning of the document, and the like).

### Example Method for Secure Chat Interactions

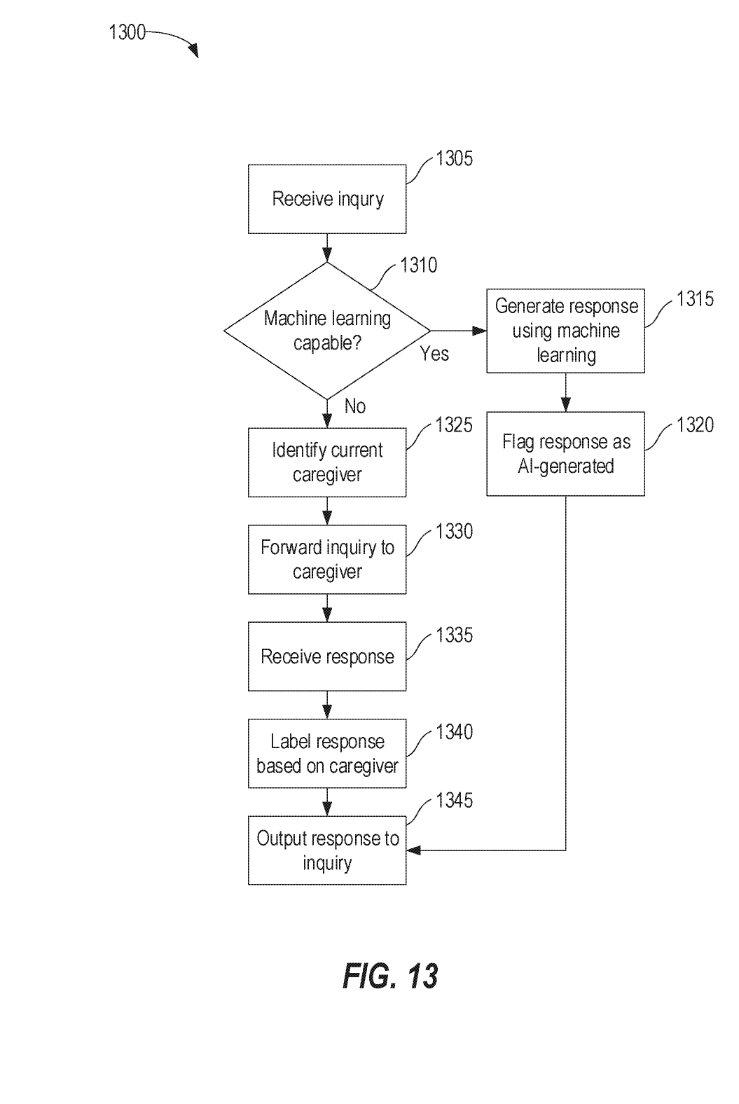

FIG. 13 is a flow diagram depicting an example method 1300 for secure chat interactions, according to some embodiments of the present disclosure. In some embodiments, the method 1300 is performed by an application system, such as the application system 105 of FIG. 1. For example, some or all of the method 1300 may be performed or implemented using a healthcare application, such as the healthcare applications 120 of FIG. 1, and/or using one or more GUIs, such as the interfaces discussed above with reference to FIGS. 2-9.

At block 1305, the application system receives an inquiry (e.g., from a user). For example, as discussed above with reference to the interface 400 of FIG. 4, the user may initiate a chat by entering a question or inquiry. In some embodiments, the inquiry may be unstructured (e.g., natural language), and may be provided as text, as an audio recording, and the like. In some embodiments, as discussed above, the user may specify the context of the inquiry. For example they may indicate that the inquiry relates to nursing, social work, billing, and the like. In some embodiments, the application system may infer or determine the context automatically. For example, the application system may evaluate some or all of the inquiry using machine learning and/or NLP to infer the context. As another example, the application system may determine the context based on how the user triggered the request. For example, if the user initiated the inquiry based on a specific entry on an activity timeline, the application system may determine that this entry is the relevant context for the inquiry.

At block 1310, the application system determines whether the available machine learning model(s) or system(s) are capable of responding to the inquiry. For example, as discussed above with reference to FIG. 6, the application system may determine whether the request relates to a specific and clearly defined piece of information (such as the resident’s vitals, an invoice amount or due date, and the like), or otherwise fits within one or more defined categories that are suitable for machine learning.

If so, the method 1300 continues to block 1315, where the application system generates a response using one or more machine learning models. For example, the application system may process the inquiry using a language model (e.g., an LLM) in a retrieval-augmented generation (RAG) process to automatically generate a response leveraging data contained in the various repositories (depending on the particular content of the request). For example, as discussed above, the application system may evaluate the EHR to find the resident’s most recent vitals, or may evaluate various documents to determine the next due date for the resident’s invoices.

At block 1320, the application system flags or labels the response as AI-generated (e.g., by indicating that the answer was provided by an AI ChatBot, as discussed above), and the method 1300 continues to block 1345, discussed in more detail below. Returning to block 1310, if the application system determines that the inquiry cannot (or should not) be answered using machine learning, the method 1300 continues to block 1325.

At block 1325, the application system identifies the current caregiver assigned to the resident (or other relevant individual to respond to the inquiry). For example, as discussed above, the application system may evaluate staffing (such as the staffing 112 of FIG. 1) to determine which caregiver(s) are assigned to the resident, and/or which caregiver(s) is best suited to respond, such as based on relative caregiver workloads, based on who is most relevant to the context or content of the request (e.g., which caregiver prescribed the medication about which the user is asking), and the like.

At block 1330, the application system forwards the inquiry to the identified caregiver for response. For example, as discussed above, the application system may provide the inquiry via a healthcare application executing on the caregiver’s mobile device.

At block 1335, the application system receives a response from the caregiver. At block 1340, the application system then labels the response to indicate the caregiver that provided it (e.g., with the caregivers name, title, and the like0.

At block 1345, the application system can then output the response to the inquiry, labeled based on who (or what) generated the response, as discussed above. For example, the application system may output the response via a GUI of a healthcare application executing on the user’s device (e.g., the interface 500 of FIG. 5 and/or the interface 600 of FIG. 6).

In this way, by dynamically switching between AI-based output and human-based output, the application system can ensure that the responses provided are accurate and reliable. Further, by refraining from using the machine learning model(s) to process the inquiry in some cases, the application system can significantly reduce the computational expense of the response process (e.g., as compared to conventional approaches that attempt to use machine learning for all responses).

### Example Method for Improved Healthcare via Data Unification

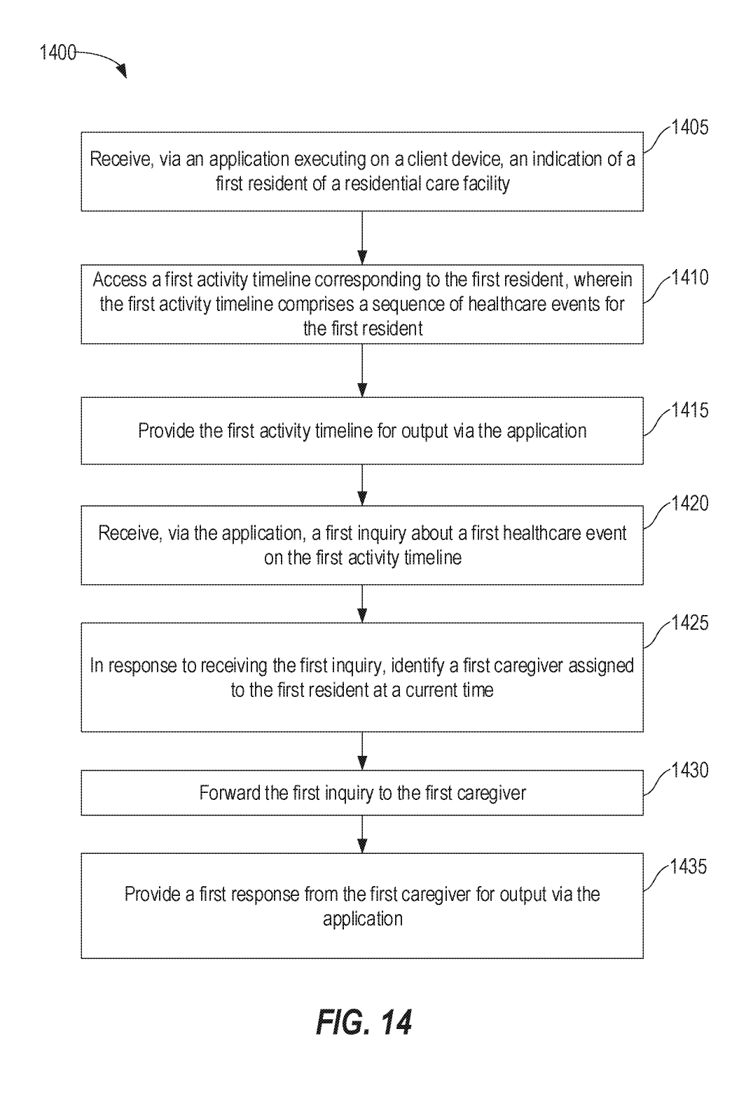

FIG. 14 is a flow diagram depicting an example method 1400 for improved healthcare via data unification, according to some embodiments of the present disclosure. In some embodiments, the method 1400 is performed by an application system, such as the application system 105 of FIG. 1. For example, some or all of the method 1400 may be performed or implemented using a healthcare application, such as the healthcare applications 120 of FIG. 1, and/or using one or more GUIs, such as the interfaces discussed above with reference to FIGS. 2-9.

At block 1405, an indication of a first resident of a residential care facility is received via an application (e.g., the healthcare application 120A of FIG. 1) executing on a client device (e.g., the client device 115 of FIG. 1).

At block 1410, a first activity timeline (e.g., depicted in the portion 305 of FIG. 3) corresponding to the first resident is accessed, wherein the first activity timeline comprises a sequence of healthcare events (e.g., events 330 of FIG. 3) for the first resident.

At block 1415, the first activity timeline is provided for output via the application.

At block 1420, a first inquiry about a first healthcare event on the first activity timeline is received via the application (e.g., via the portion 435 of FIG. 4).

At block 1425, in response to receiving the first inquiry, a first caregiver assigned to the first resident at a current time is identified (e.g., based on the staffing 112 of FIG. 1).

At block 1430, the first inquiry is forwarded to the first caregiver.

At block 1435, a first response (e.g., the message 525 of FIG. 5) from the first caregiver is provided for output via the application.

### Example Processing System for Data Unification and Sharing

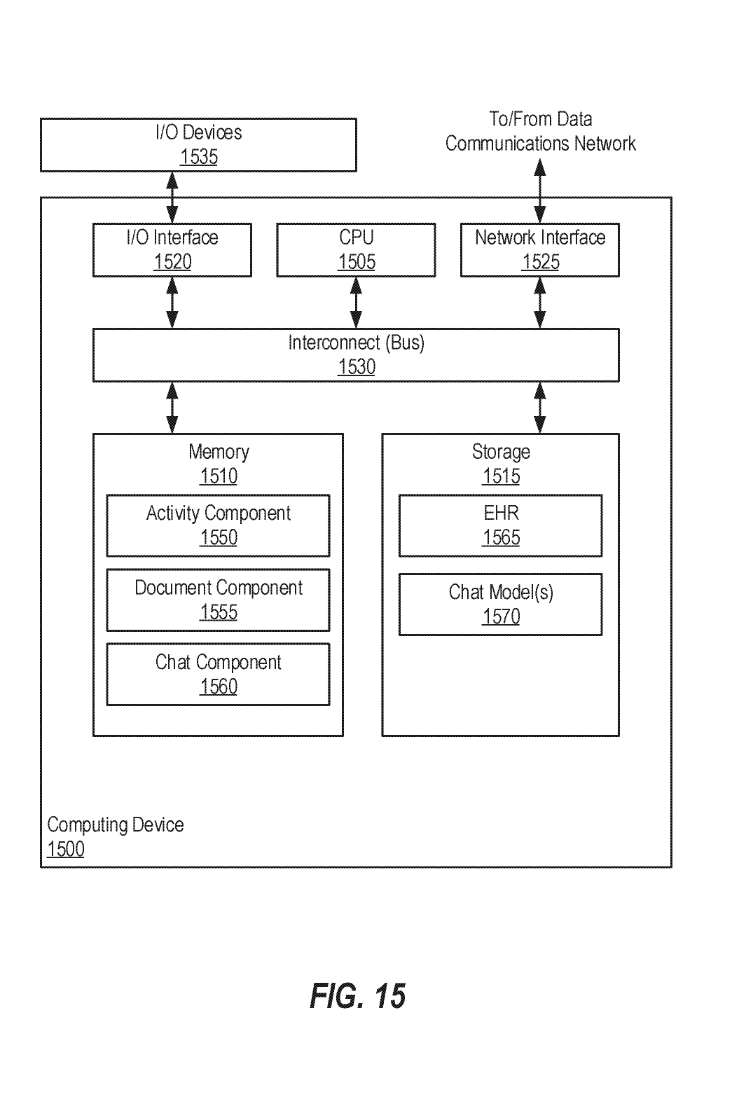

FIG. 15 depicts an example computing device configured to perform various aspects of the present disclosure, according to some embodiments disclosed herein.

FIG. 15 depicts an example computing device 1500 configured to perform various aspects of the present disclosure, according to some embodiments disclosed herein. Although depicted as a physical device, in embodiments, the computing device 1500 may be implemented using virtual device(s), and/or across a number of devices (e.g., in a cloud environment). In one embodiment, the computing device 1500 corresponds to any element or aspect of an application system, such as the application system 105 discussed above with reference to FIGS. 1-14, and/or a healthcare application, such as the healthcare applications 120 discussed above with reference to FIGS. 1-14.

As illustrated, the computing device 1500 includes a CPU 1505, memory 1510, storage 1515, a network interface 1525, and one or more input/output (I/O) interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes an activity component 1550, a document component 1555, and a chat component 1560, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software. Additionally, in some embodiments, additional components may be present, such as training components for training or refining the machine learning model(s).

In some embodiments, the activity component 1550 can be used to evaluate EHR data to generate activity timelines for residents, as discussed above. For example, the activity component 1550 may identify relevant events and generate corresponding timelines based on details extracted from the medical records of the resident, as discussed above.

In some embodiments, the document component 1555 can be used to manage secure document access and updates, as discussed above. For example, the document component 1555 may provide selective access to documents as needed, and may further enable electronic signature of such documents and storage of such signed documents in accordance with various regulations or policies, as discussed above.

In some embodiments, the chat component 1560 can be used to manage and/or provide chat or conversation functionality, as discussed above. For example, the chat component 1560 may dynamically determine whether to use machine learning to respond to a given inquiry. If so, the chat component 1560 may use machine learning to generate the response. If not, the chat component 1560 may identify a relevant caregiver (or other user) for the chat, and may thereafter forward the request to the identified user, as discussed above.

In the illustrated example, the storage 1515 includes EHR 1565 and one or more chat models 1570. The EHR 1565 may generally correspond to electronic health-related records of one or more residents in one or more residential care facilities, such as the EHR 110 of FIG. 1. In some embodiments, the chat models 1570 may correspond to language models (e.g., LLMs) used to provide textual responses to user inquiries (when appropriate), as discussed above. Although depicted as residing in storage 1515, the depicted data may be stored in any suitable location, including memory 1510.

Generally, the depicted components (and others not depicted) in memory 1510 may be used to implement one or more embodiments discussed above.

### Example Clauses

Clause 1: A method, comprising: receiving, via an application executing on a client device, an indication of a first resident of a residential care facility; accessing a first activity timeline corresponding to the first resident, wherein the first activity timeline comprises a sequence of healthcare events for the first resident; providing the first activity timeline for output via the application; receiving, via the application, a first inquiry about a first healthcare event on the first activity timeline; in response to receiving the first inquiry, identifying a first caregiver assigned to the first resident at a current time; forwarding the first inquiry to the first caregiver; and providing a first response from the first caregiver for output via the application.

Clause 2: A method according to Clause 1, further comprising: providing a first document for signature via the application; receiving, via the application, a signature for the first document; and updating one or more healthcare records for the first resident based on the signature.

Clause 3: A method according to any of Clauses 1-2, wherein accessing the first activity timeline comprises: accessing the healthcare events from one or more electronic healthcare record (EHR) repositories; and generating the first activity timeline by ordering the healthcare events in sequence.

Clause 4: A method according to any of Clauses 1-3, wherein: the first inquiry comprises natural language text, and forwarding the first inquiry to the first caregiver is performed in response to determining that a machine learning (ML) model trained to respond to user inquiries cannot generate an acceptable response to the first inquiry.

Clause 5: A method according to any of Clauses 1-4, further comprising: receiving a second inquiry comprising natural language text; generating a second response based on processing the second inquiry using a machine learning (ML) model trained to respond to user inquiries; and in response to determining that the second response satisfies one or more criteria: refraining from forwarding the second response to a caregiver; and providing the second response and a flag indicating that the second response was generated using ML for output via the application.

Clause 6: A method according to any of Clauses 1-5, further comprising, in response to determining that a new healthcare event for the first resident occurred: updating the first activity timeline to include the new healthcare event; and providing the updated first activity timeline for output via the application.

Clause 7: A method according to any of Clauses 1-6, further comprising: receiving, via the application, an indication of a second resident; receiving, via the application, a second inquiry about a second healthcare event on a second activity timeline of the second resident; in response to receiving the second inquiry, identifying a second caregiver assigned to the second resident; and forwarding the second inquiry to the second caregiver.

Clause 8: A system, comprising: a memory comprising computer-executable instructions; and one or more processors configured to execute the computer-executable instructions and cause the processing system to perform a method in accordance with any one of Clauses 1-7.

Clause 9: A system, comprising means for performing a method in accordance with any one of Clauses 1-7.

Clause 10: A non-transitory computer-readable medium comprising computer-executable instructions that, when executed by one or more processors of a processing system, cause the processing system to perform a method in accordance with any one of Clauses 1-7.

Clause 11: A computer program product embodied on a computer-readable storage medium comprising code for performing a method in accordance with any one of Clauses 1-7.

### Additional Considerations

One or more elements or aspects or steps, or any portion(s) thereof, from one or more of any of claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present invention, a user may access applications or systems (e.g., the evaluation system and/or intervention system) or related data available in the cloud. For example, the evaluation system could execute on a computing system in the cloud and train and use machine learning models to predict various user health information, as discussed above. In such a case, the evaluation system could receive and process sensor data, and store the models and predictions at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
receiving, via an application executing on a client device, an indication of a first resident of a residential care facility;
accessing a first activity timeline corresponding to the first resident, wherein the first activity timeline comprises a sequence of healthcare events for the first resident;
providing the first activity timeline for output via the application;
receiving, via the application, a first inquiry about a first healthcare event on the first activity timeline;
in response to receiving the first inquiry, identifying a first caregiver assigned to the first resident at a current time;
forwarding the first inquiry to the first caregiver; and
providing a first response from the first caregiver for output via the application.

2. The method of claim 1, further comprising:
providing a first document for signature via the application;
receiving, via the application, a signature for the first document; and
updating one or more healthcare records for the first resident based on the signature.

3. The method of claim 1, wherein accessing the first activity timeline comprises:
accessing the healthcare events from one or more electronic healthcare record (EHR) repositories; and
generating the first activity timeline by ordering the healthcare events in sequence.

4. The method of claim 1, wherein:
the first inquiry comprises natural language text, and
forwarding the first inquiry to the first caregiver is performed in response to determining that a machine learning (ML) model trained to respond to user inquiries cannot generate an acceptable response to the first inquiry.

5. The method of claim 1, further comprising:
receiving a second inquiry comprising natural language text;
generating a second response based on processing the second inquiry using a machine learning (ML) model trained to respond to user inquiries; and
in response to determining that the second response satisfies one or more criteria:
refraining from forwarding the second response to a caregiver; and
providing the second response and a flag indicating that the second response was generated using ML for output via the application.

6. The method of claim 1, further comprising, in response to determining that a new healthcare event for the first resident occurred:
updating the first activity timeline to include the new healthcare event; and
providing the updated first activity timeline for output via the application.

7. The method of claim 1, further comprising:
receiving, via the application, an indication of a second resident;
receiving, via the application, a second inquiry about a second healthcare event on a second activity timeline of the second resident;
in response to receiving the second inquiry, identifying a second caregiver assigned to the second resident; and
forwarding the second inquiry to the second caregiver.

8. One or more non-transitory computer-readable media collectively or individually comprising computer-executable instructions that, when executed by one or more processors of one or more processing systems, cause the one or more processing systems to collectively or individually perform an operation comprising:
receiving, via an application executing on a client device, an indication of a first resident of a residential care facility;
accessing a first activity timeline corresponding to the first resident, wherein the first activity timeline comprises a sequence of healthcare events for the first resident;
providing the first activity timeline for output via the application;
receiving, via the application, a first inquiry about a first healthcare event on the first activity timeline;
in response to receiving the first inquiry, identifying a first caregiver assigned to the first resident at a current time;
forwarding the first inquiry to the first caregiver; and
providing a first response from the first caregiver for output via the application.

9. The one or more non-transitory computer-readable media of claim 8, the operation further comprising:
providing a first document for signature via the application;
receiving, via the application, a signature for the first document; and
updating one or more healthcare records for the first resident based on the signature.

10. The one or more non-transitory computer-readable media of claim 8, wherein accessing the first activity timeline comprises:
accessing the healthcare events from one or more electronic healthcare record (EHR) repositories; and
generating the first activity timeline by ordering the healthcare events in sequence.

11. The one or more non-transitory computer-readable media of claim 8, wherein:
the first inquiry comprises natural language text, and
forwarding the first inquiry to the first caregiver is performed in response to determining that a machine learning (ML) model trained to respond to user inquiries cannot generate an acceptable response to the first inquiry.

12. The one or more non-transitory computer-readable media of claim 8, further comprising:
receiving a second inquiry comprising natural language text;
generating a second response based on processing the second inquiry using a machine learning (ML) model trained to respond to user inquiries; and
in response to determining that the second response satisfies one or more criteria:
refraining from forwarding the second response to a caregiver; and
providing the second response and a flag indicating that the second response was generated using ML for output via the application.

13. The one or more non-transitory computer-readable media of claim 8, further comprising, in response to determining that a new healthcare event for the first resident occurred:
updating the first activity timeline to include the new healthcare event; and
providing the updated first activity timeline for output via the application.

14. The one or more non-transitory computer-readable media of claim 8, further comprising:
receiving, via the application, an indication of a second resident;
receiving, via the application, a second inquiry about a second healthcare event on a second activity timeline of the second resident;
in response to receiving the second inquiry, identifying a second caregiver assigned to the second resident; and
forwarding the second inquiry to the second caregiver.

15. A system, comprising:
one or more memories collectively or individually comprising computer-executable instructions; and
one or more processors configured to, individually or collectively, execute the computer-executable instructions and cause the system to perform an operation comprising:
receiving, via an application executing on a client device, an indication of a first resident of a residential care facility;
accessing a first activity timeline corresponding to the first resident, wherein the first activity timeline comprises a sequence of healthcare events for the first resident;
providing the first activity timeline for output via the application;
receiving, via the application, a first inquiry about a first healthcare event on the first activity timeline;
in response to receiving the first inquiry, identifying a first caregiver assigned to the first resident at a current time;
forwarding the first inquiry to the first caregiver; and
providing a first response from the first caregiver for output via the application.

16. The system of claim 15, the operation further comprising:
providing a first document for signature via the application;
receiving, via the application, a signature for the first document; and
updating one or more healthcare records for the first resident based on the signature.

17. The system of claim 15, wherein accessing the first activity timeline comprises:
accessing the healthcare events from one or more electronic healthcare record (EHR) repositories; and
generating the first activity timeline by ordering the healthcare events in sequence.

18. The system of claim 15, wherein:
the first inquiry comprises natural language text, and
forwarding the first inquiry to the first caregiver is performed in response to determining that a machine learning (ML) model trained to respond to user inquiries cannot generate an acceptable response to the first inquiry.

19. The system of claim 15, further comprising:
receiving a second inquiry comprising natural language text;
generating a second response based on processing the second inquiry using a machine learning (ML) model trained to respond to user inquiries; and
in response to determining that the second response satisfies one or more criteria:
refraining from forwarding the second response to a caregiver; and
providing the second response and a flag indicating that the second response was generated using ML for output via the application.

20. The system of claim 15, further comprising:
receiving, via the application, an indication of a second resident;
receiving, via the application, a second inquiry about a second healthcare event on a second activity timeline of the second resident;
in response to receiving the second inquiry, identifying a second caregiver assigned to the second resident; and
forwarding the second inquiry to the second caregiver.
